(19) United States
(12) Patent Application Publication
(10) Pub. No.: US 2024/0206956 A1
(43) Pub. Date: Jun. 27, 2024

(54) TREATMENT TOOL FOR ENDOSCOPE

(71) Applicant: OLYMPUS MEDICAL SYSTEMS CORP., Tokyo (JP)

(72) Inventors: Shunsuke MOTOSUGI, Hachioji-shi (JP); Yusuke SHIOTA, Machida-shi (JP); Hiromasa KATO, Tokyo (JP); Yuya MATSUOKA, Akishima-shi (JP); Kazuki OZAWA, Hachioji-shi (JP)

(21) Appl. No.: 18/390,708

(22) Filed: Dec. 20, 2023

Related U.S. Application Data
(60) Provisional application No. 63/476,800, filed on Dec. 22, 2022.
(60) Provisional application No. 63/487,426, filed on Feb. 28, 2023.

(51) Int. Cl. A61B 18/14; A61B 17/32; A61B 17/34

(52) U.S. Cl. CPC A61B 18/1482; A61B 17/320016; A61B 17/3478; A61B 2017/00269

(57) ABSTRACT

A treatment tool for an endoscope includes a sheath having a distal end and a proximal end, an electrode provided on a distal end portion of the sheath, and a rod provided at a position away from the electrode in a radial direction. The rod configured to move forward and advance, and, in a state in which at least the electrode and the rod protrude from the sheath, a part of a flow path through which a fluid passes is formed in at least a part of a space between the electrode and the rod.

### CROSS-REFERENCE TO RELATED APPLICATION

Priority is claimed to U.S. Patent Provisional Application No. 63/476,800, filed Dec. 22, 2022, and U.S. Patent Provisional Application No. 63/487,426, filed Feb. 28, 2023, the contents of which are incorporated herein by reference.

### TECHNICAL FIELD

The present disclosure relates to a treatment tool for an endoscope.

### BACKGROUND

In endoscopic therapy such as endoscopic submucosal dissection (ESD) or the like, a treatment tool for an endoscope used in incision or exfoliation such as a high frequency knife or the like, a treatment tool for an endoscope used in local injection, or the like, may be used.

Examples of a high frequency knife for an endoscope are discussed in Patent Document 1 and Patent Document 2 can perform an incision or exfoliation treatment and/or a local injection treatment.

### PATENT DOCUMENTS

[Patent Document 1] Chinese Utility Model Registration No. 211355353
[Patent Document 2] Chinese Utility Model Registration No. 209032622

### SUMMARY

The present disclosure is directed to providing a treatment tool for an endoscope capable of appropriately performing incision or exfoliation treatments or local injection treatments and is easier to handle than a treatment tool utilizing a cylindrical needle and rod-shaped knife (which makes an outer diameter of the sheath larger) such as in Patent Documents 1 and 2, for example.

A treatment tool for use with an endoscope according to a first aspect of the present disclosure may include a sheath having a distal end and a proximal end, an electrode provided on a distal end portion of the sheath, and a rod provided at a position away from the electrode in a radial direction to move forward and advance. In a state in which at least the electrode and the rod protrude from the sheath, a part of a flow path through which a fluid passes can be formed in at least a part of a space between the electrode and the rod.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a general view of an endoscope treatment system according to a first embodiment.

FIG. 2 is a general view showing a treatment tool of the endoscope treatment system.

FIG. 3 is a perspective view of a distal end portion of the treatment tool.

FIG. 4 is a cross-sectional view of the distal end portion of the treatment tool when a distal end of a knife is located at a first position.

FIG. 5 is a cross-sectional view of the distal end portion of the treatment tool when the distal end of the knife is located at a second position.

FIG. 6 is a front view of the distal end portion of the treatment tool when seen in a direction along a longitudinal direction.

FIG. 7 is a cross-sectional view of the distal end portion of the treatment tool when a sharp member is located at a distal end position.

FIG. 8 is a cross-sectional view along line C0-C0 shown in FIG. 7.

FIG. 9 is a cross-sectional view showing a variant of the treatment tool.

FIG. 10 is a cross-sectional view showing the variant.

FIG. 11 is a cross-sectional view showing the variant.

FIG. 12 is a perspective view of a distal end portion of a treatment tool according to a second embodiment.

FIG. 13 is a cross-sectional view of the distal end portion of the treatment tool.

FIG. 14 is a cross-sectional view of the distal end portion of the treatment tool.

FIG. 15 is a cross-sectional view of the distal end portion of the treatment tool.

FIG. 16 is a cross-sectional view along line C1-C1 shown in FIG. 15.

FIG. 17 is a cross-sectional view showing a variant of the treatment tool.

FIG. 18 is a cross-sectional view showing the variant.

FIG. 19 is a cross-sectional view showing the variant.

FIG. 20 is a perspective cross-sectional view showing a counterflow prevention member.

FIG. 21 is a cross-sectional view along line C2-C2 shown in FIG. 20.

FIG. 22 is a perspective view of a distal end portion of a treatment tool according to a third embodiment.

FIG. 23 is a cross-sectional view of the distal end portion of the treatment tool.

FIG. 24 is a cross-sectional view of the distal end portion of the treatment tool.

FIG. 25 is a cross-sectional view of the distal end portion of the treatment tool.

FIG. 26 is a cross-sectional view along line C3-C3 shown in FIG. 24.

FIG. 27 is a perspective cross-sectional view showing a variant of the treatment tool.

FIG. 28 is a perspective cross-sectional view showing the variant.

FIG. 29 is a cross-sectional view along line C4-C4 shown in FIG. 28.

FIG. 30 is a perspective view of a distal end portion of a treatment tool according to a fourth embodiment.

FIG. 31 is a perspective view of the distal end portion of the treatment tool.

FIG. 32 is a cross-sectional view of the distal end portion of the treatment tool.

FIG. 33 is a cross-sectional view of the distal end portion of the treatment tool.

FIG. 34 is a cross-sectional view of the distal end portion of the treatment tool.

FIG. 35 is a cross-sectional view along line C5-C5 shown in FIG. 34.

FIG. 36 is a cross-sectional view showing a variant of the treatment tool.

FIG. 37 is a cross-sectional view showing the variant.

FIG. 38 is a cross-sectional view showing the variant.

FIG. 39 is a perspective view of a distal end portion of a treatment tool according to a fifth embodiment.

FIG. 40 is a cross-sectional view of the distal end portion of the treatment tool.

FIG. 41 is a cross-sectional view of the distal end portion of the treatment tool.

FIG. 42 is a cross-sectional view of the distal end portion of the treatment tool.

FIG. 43 is a perspective view showing a variant of the treatment tool.

FIG. 44 is a cross-sectional view of the distal end portion of the variant.

FIG. 45 is a cross-sectional view of the distal end portion of the variant.

FIG. 46 is a cross-sectional view of the distal end portion of the variant.

FIG. 47 is a view showing a variant of the operation unit.

FIG. 48 is a view showing a variant of the operation unit.

FIG. 49 is a view showing the variant.

FIG. 50 is a view showing a variant of the operation unit.

FIG. 51 is a view showing the variant.

### DETAILED DESCRIPTION

### First Embodiment

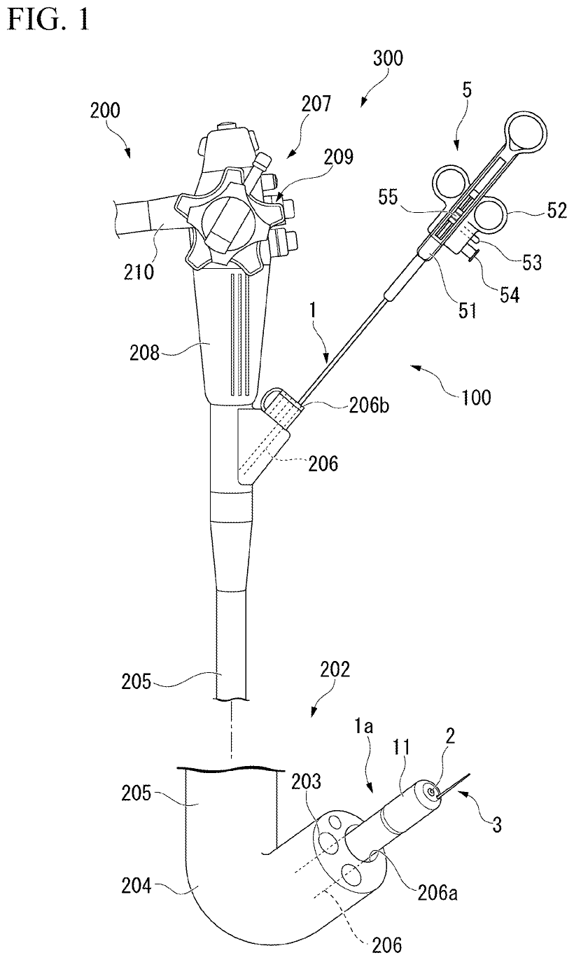

An endoscope treatment system 300 according to a first embodiment of the present disclosure will be described with reference to FIG. 1 to FIG. 7. FIG. 1 is a general view of the endoscope treatment system 300 according to the embodiment.

### [Endoscope Treatment System 300]

As shown in FIG. 1, the endoscope treatment system 300 includes an endoscope 200 and a treatment tool 100. The treatment tool 100 may be used by being inserted into the endoscope 200.

### [Endoscope 200]

The endoscope 200 may be a flexible endoscope and may include an insertion part 202 inserted into a body from a distal end, and an operation unit 207 attached to a proximal end of the insertion part 202.

The insertion part 202 may include an imaging part 203, a curved portion 204, and a flexible portion 205. The imaging part 203, the curved portion 204 and the flexible portion 205 may be disposed in sequence from the distal end of the insertion part 202. A channel 206 into which the treatment tool 100 can be inserted is provided in the insertion part 202. A distal end opening portion 206a of the channel 206 may be provided in the distal end of the insertion part 202.

The imaging part 203 may include an imaging element such as a charge coupled device (CCD), a complementary metal oxide semiconductor (CMOS), or the like, and can image an area that is a treatment target. The imaging part 203 can image a knife 2 of the treatment tool 100 in a state in which the treatment tool 100 protrudes from the distal end opening portion 206a of the channel 206.

The curved portion 204 may be curved according to the operation of the operation unit 207 by an operator. The flexible portion 205 may include a tubular area having flexibility.

The operation unit 207 may be connected to the flexible portion 205. The operation unit 207 may include a grip 208, an input unit 209, a proximal end opening portion 206b of the channel 206, and a universal cord 210. The grip 208 can include an area gripped by an operator. The input unit 209 may receive (or be configured to receive) an operation input to curve the curved portion 204. The universal cord 210 may output the image imaged by the imaging part 203 to the outside. The universal cord 210 may be connected to a display device such as a liquid crystal display (LCD) or the like via an image processing device including a processor or the like.

### [Treatment Tool 100]

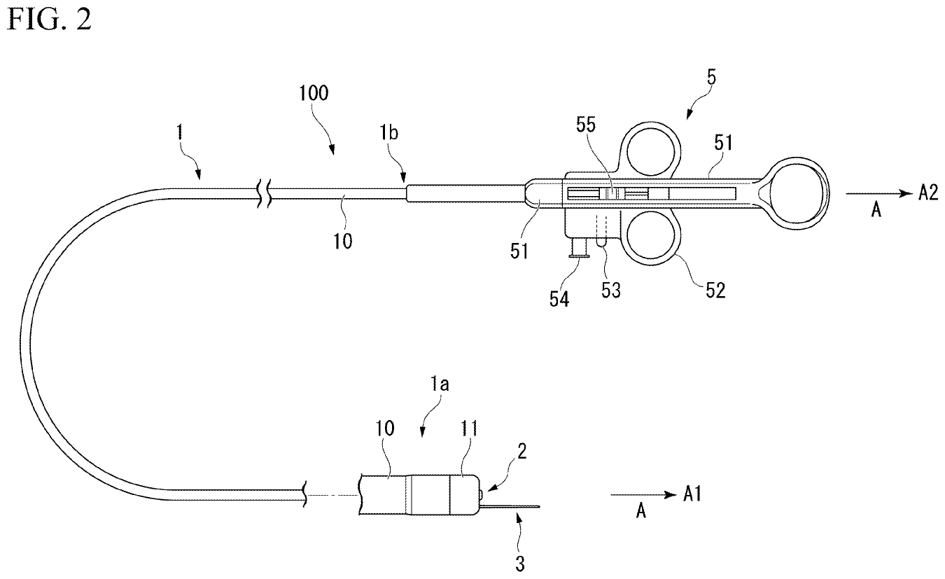

FIG. 2 is a general view showing the treatment tool 100.

The treatment tool (treatment tool for an endoscope) 100 may include a sheath 1, the knife 2, a sharp member 3, an operation wire 4 (see FIG. 4), and an operation unit 5. The operation wire 4 may include a first operation wire 41 connected to the knife 2, and a second operation wire 42 connected to the sharp member 3. In the following description, in a longitudinal direction A of the treatment tool 100, a side inserted into a patient's body is referred to as “a distal end side (distal side) A1,” and a side of the operation unit is referred to as “a proximal end side (proximal side) A2.”

The sheath 1 may be an elongate tubular member extending from a distal end 1a to a proximal end 1b. The sheath 1 can include an outer diameter that enables insertion into the channel 206 of the endoscope 200 and can move forward and rearward in the channel 206. As shown in FIG. 1, in a state in which the sheath 1 can be inserted into the channel 206, the distal end 1a of the sheath 1 can protrude from and be inserted into the distal end opening portion 206a of the channel 206.

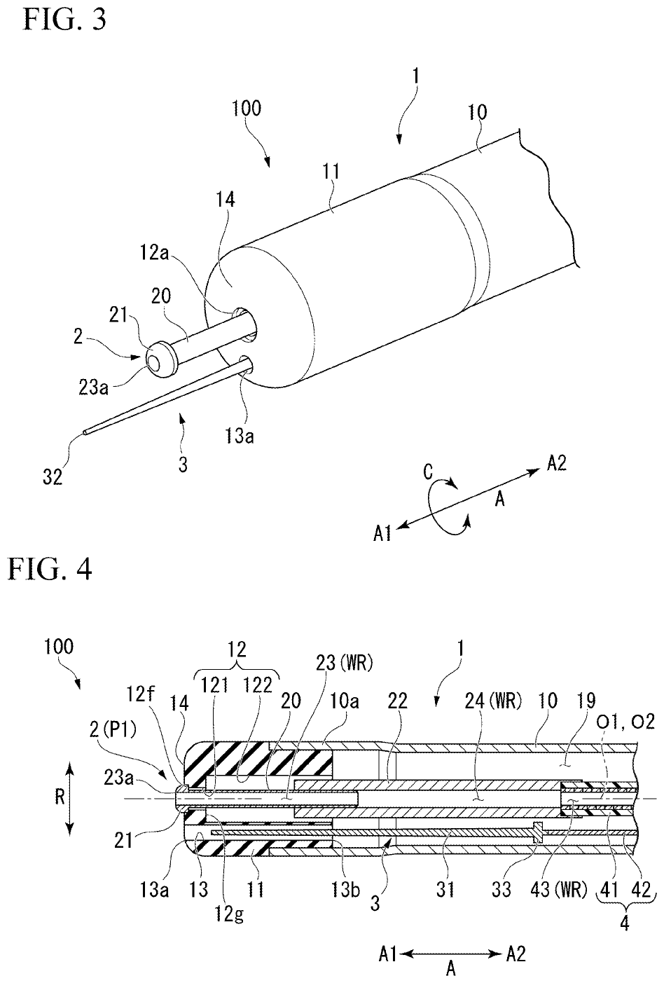

FIG. 3 is a perspective view of the distal end portion of the treatment tool 100. The sheath 1 may include a tube 10 extending in the longitudinal direction A, and a distal end member 11 provided on a distal end of the tube 10. Further, the tube 10 and the distal end member 11 of the sheath 1 may be formed integrally with each other.

FIG. 4 is a cross-sectional view of the distal end portion of the treatment tool 100 when the distal end of the knife 2 is located at a first position P1.

The tube 10 may be an elongate tubular member having flexibility and insulation. The tube may be formed from or made of a resin or any similar material. In an example, a diameter expansion part (distal end portion) 10a having an outer diameter greater than that of the proximal end side may be provided on the distal end of the tube 10. The diameter expansion part (distal end portion) 10a does not necessarily have to include or be formed such that it has an outer diameter greater than that of the tube 10. For example, the diameter expansion part may have the same outer diameter as that of the tube 10. The distal end member 11 may be fitted and attached into the diameter expansion part 10a. Further, the distal end member 11 may be adhered to the diameter expansion part 10a by an adhesive agent or the like.

The distal end member 11 may be formed in a cylindrical or substantially cylindrical shape. For example, “cylindrical shape” can include a shape close to a cylindrical shape, in addition to a strict cylindrical shape. The distal end member 11 may be formed from, made of, or include an insulating material such as a resin or the like. The distal end member 11 may be formed to have a first through-hole 12 and a second through-hole 13.

The first through-hole 12 may be a hole provided in the distal end member 11 and passing through the distal end member 11 in the longitudinal direction A. A distal end of the first through-hole 12 may be in communication or engaged with a first opening 12a formed in a distal end surface 14 of the distal end member 11. A proximal end of the first through-hole 12 may be in communication or engaged with an internal space 19 of the tube 10. The knife 2 can be inserted through the first through-hole 12.

The first through-hole 12 may include a first region 121 on the distal end side A1, and a second region 122 (see FIG. 16) on the proximal end side A2. The first region 121 may include a part of the through-hole passing through in the longitudinal direction A. In an example, a cross section of the first region perpendicular to the longitudinal direction A may have a circular or substantially circular shape. The second region 122 may include a part of the through-hole passing through in the longitudinal direction A. In an example, a cross section of the second region perpendicular to the longitudinal direction A may have a circular or substantially circular shape. The first region 121 and the second region 122 may communicate, engage, connect, or the like with respect to each other. An inner diameter of the first region 121 may be smaller than that of the second region 122. A step difference 12g may be formed between the first region 121 and the second region 122.

A flange storage part 12f may be configured to accommodate a flange 21 of the knife 2 and may be formed in the first opening 12a of the first through-hole 12 of the distal end member 11. When the knife 2 is accommodated in the sheath 1 and the flange 21 is accommodated in the flange storage part 12f, (i.e., when the flange 21 abuts the flange storage part 12f), the distal end of the knife 2 can protrude closer to the distal end side A1 than the distal end surface 14 of the distal end member 11. Further, the flange storage part 12f may not be formed in the first opening 12a and may be formed at another, appropriate or desired location.

The second through-hole 13 may be a hole provided in the distal end member 11 and passing through the distal end member 11 in the longitudinal direction A. The distal end of the second through-hole 13 may be in communication with, engaged with, connected to or the like a second opening 13a formed in the distal end surface 14 of the distal end member 11. A proximal end of the second through-hole 13 may be in communication with, engaged with, connected to, or the like the internal space 19 of the tube 10. The sharp member 3 may be inserted through the second through-hole 13.

The second opening 13a of the second through-hole 13 may be located or disposed to be separated from the first opening 12a of the first through-hole 12 in a radial direction R. In an example, the first through-hole 12 does not communicate or engage directly with (or connect directly to) the second through-hole 13.

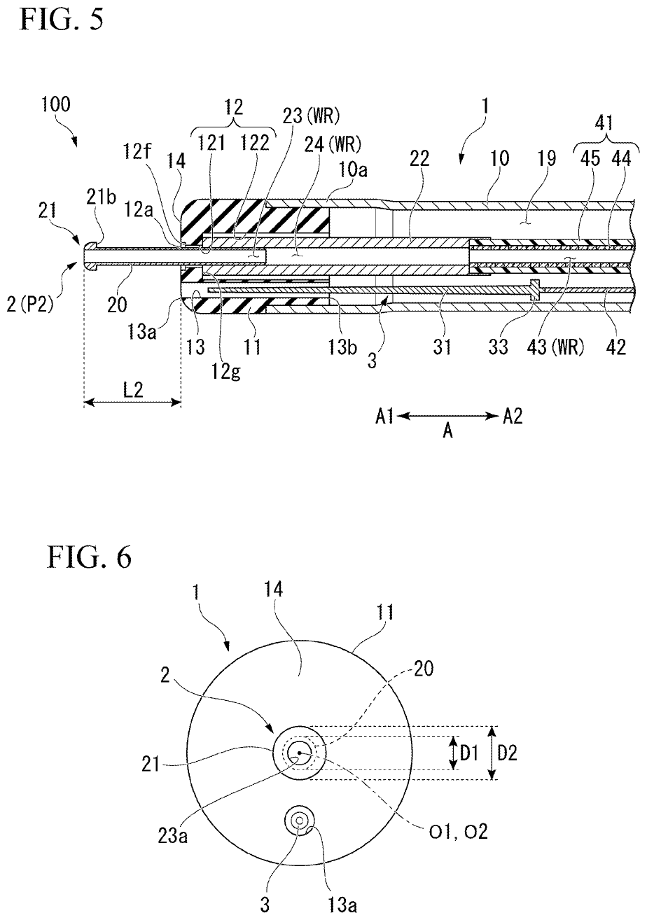

FIG. 5 is a cross-sectional view of the distal end portion of the treatment tool 100 when the distal end of the knife 2 is located at a second position P2.

The knife (electrode) 2 may be a round rod-shaped member formed of a metal. In an example, the knife may include a shape close to a round rod shape, in addition to a strict round rod shape. The knife 2 may be formed of a material such as stainless steel or the like. The knife 2 has conductivity and can be energized with high frequency current. The knife 2 may include a knife main body 20, the flange 21, and a connecting member 22.

The knife 2 may be inserted through the first through-hole 12 of the distal end member 11 of the sheath 1 in the longitudinal direction A and may protrude (or be configured to protrude) from and be inserted into the first opening 12a on the distal end side A1. Further, the knife 2 may be fixed such that it cannot be advanced or retracted while protruding from the first opening 12a on the distal end side A1.

A center axis O2 of the knife 2 in the longitudinal direction A may match a center axis O1 of the sheath 1 in the longitudinal direction A. Further, “match” includes not only an aspect of exactly matching but also an aspect of almost matching.

The knife main body 20 may be a round rod-shaped member formed of a metal. An outer diameter of the knife main body 20 may be smaller than an inner diameter of the first region 121. The knife main body 20 can move (or be configured to move) in the first region 121 forward and rearward in the longitudinal direction A.

FIG. 6 is a front view of the distal end portion of the treatment tool 100 when seen in a direction along the longitudinal direction A.

The flange (distal end diameter expansion part) 21 may be a disk-shaped conductive member provided on the distal end of the knife main body 20. When seen or viewed in a front view viewed from a direction along the longitudinal direction A, an outer circumference of the flange 21 may be formed in a circular shape concentric with the outer circumference of the knife main body 20. A length D2 of the flange 21 in the radial direction R perpendicular to the longitudinal direction A may be greater than a length D1 of the knife main body 20 in the radial direction R. A planar proximal end surface 21b may be formed on the proximal end side A2 of the flange 21. Further, the flange 21 may not limited to a disk shape and may have a triangular shape or a hook shape.

As shown in FIG. 4, when the knife 2 is retracted from the sheath 1, the proximal end surface 21b of the knife 2 comes into contact with the flange storage part 12f of the distal end member 11. As the proximal end surface 21b of the knife 2 comes into contact with the flange storage part 12f of the distal end member 11, the distal end of the knife 2 can be positioned at the first position P1 that is a position closest to the proximal end side A2.

The knife main body 20 and the flange 21 may include a first pipeline 23 extending in the longitudinal direction A. The first pipeline 23 may be in communication with, engaged with, connected to, or the like a distal end opening 23a formed in the flange 21. The distal end opening 23a may be an opening provided in the distal end side A1 of the flange 21.

The connecting member 22 may be a cylindrical member formed of a metal. Further, the “cylindrical shape” may include a shape close to a cylindrical shape, in addition to a strict cylindrical shape. The connecting member 22 can connect the knife main body 20 and the first operation wire 41. An outer diameter of the connecting member 22 may be greater than an inner diameter of the first region 121 and may be smaller than an inner diameter of the second region 122. In an example, the connecting member 22 cannot insert the first region 121 in the longitudinal direction A, and can move the second region 122 forward and rearward in the longitudinal direction A.

As shown in FIG. 5, when the knife 2 is advanced against the sheath 1, the distal end of the connecting member 22 can come into contact (or make contact) with the step difference 12g of the distal end member 11. As the distal end of the connecting member 22 comes into contact with the step difference 12g of the distal end member 11, the distal end of the knife 2 can be positioned at the second position P2, which is the closest position to the distal end side A1.

The connecting member 22 may include a second pipeline 24 extending in the longitudinal direction A. The second pipeline 24 may be in communication with or connect to a third pipeline 43 formed in the first pipeline 23 and the first operation wire 41.

The first operation wire 41 may be attached to a proximal end of the connecting member 22. A high frequency current can be supplied to the knife 2 via the first operation wire 41 connected to the operation unit 5. When the high frequency current is supplied from the first operation wire 41 to the knife 2, the knife main body 20 and the flange 21 can function as an active electrode configured to output a high frequency current to a biological tissue.

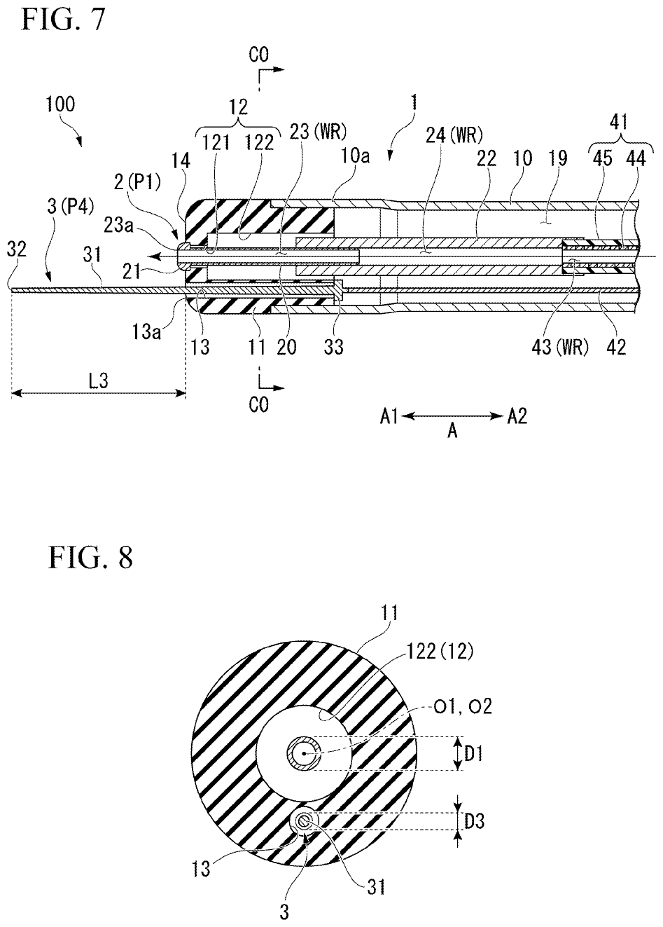

FIG. 7 is a cross-sectional view of the distal end portion of the treatment tool 100 when the sharp member 3 is located at a distal end position P4. FIG. 8 is a cross-sectional view along line C0-C0 shown in FIG. 7.

The sharp member 3 (e.g., a rod, a solid needle, or the like) may be a rod-shaped member formed of a resin material, a metal material, a ceramic material, or the like. The sharp member 3 may be a member thinner than the knife 2, and an outer diameter D3 of the sharp member 3 may be smaller than a minimum outer diameter D1 of a portion of the knife 2 that can be exposed from the distal end member 11 of the sheath 1. The sharp member 3 may include a main body portion 31, a sharp portion 32, and a flange 33.

The sharp member 3 may be inserted through the second through-hole 13 of the distal end member 11 of the sheath 1 in the longitudinal direction A, and can freely protrude from and be inserted into the second opening 13a at the distal end side A1. The sharp member 3 may be located or provided at a position spaced apart from the knife 2 in the radial direction R and may be movable forward and rearward in the longitudinal direction A. Further, the sharp member 3 may be fixed so that it cannot move forward and backward while protruding from the second opening 13a to the distal end side A1.

The main body portion 31 may be an elongated round rod-shaped member. The “round rod shape” can also include a shape close to a round rod shape, in addition to a strict round rod shape. An outer diameter of the main body portion 31 may be smaller than an inner diameter of the second through-hole 13. The main body portion 31 can move the second through-hole 13 forward and rearward in the longitudinal direction A.

The sharp portion 32 may be provided on a distal end of the main body portion 31, and the distal end side A1 is formed in a tapered shape. The sharp portion 32 can move through the second through-hole 13 forward and rearward in the longitudinal direction A.

The flange 33 (e.g., a diameter expansion part) may be provided on a proximal end of the main body portion 31. An outer diameter of at least a part of the flange 33 may be greater than an inner diameter of a proximal end opening 13b of the second through-hole 13. In an example, the flange 33 cannot be inserted through the second through-hole 13.

As shown in FIG. 7, when the sharp member 3 is moved forward against the sheath 1, the flange 33 of the sharp member 3 can come into contact with the proximal end opening 13b of the second through-hole 13 of the distal end member 11. As the flange 33 of the sharp member 3 comes into contact with the proximal end opening 13b of the second through-hole 13, the distal end of the sharp member 3 (the distal end of the sharp portion 32) can be positioned at the distal end position P4 that is a position closest to the distal end side A1.

A protrusion amount L3 of the sharp member 3 protruding from the second opening 13a when the distal end of the sharp portion 32 is disposed at the distal end position P4 (a maximum protrusion amount of the sharp member 3, see FIG. 7) may be greater than a protrusion amount L2 of the knife 2 protruding from the first opening 12a when the distal end of the knife 2 is disposed at the second position P2 (a maximum protrusion amount of the knife 2, see FIG. 5).

Further, the protrusion amount L3 of the sharp member 3 protruding from the second opening 13a when the distal end of the sharp portion 32 is disposed at the distal end position P4 may be smaller than the protrusion amount L2 of the knife 2 maximally protruding from the first opening 12a when the distal end of the knife 2 is disposed at the second position P2.

The operation wire 4 may be a wire inserted through the internal space (e.g., a pipeline, a lumen, or the like) 19 of the sheath 1. The operation wire 4 may include a first operation wire 41 and a second operation wire 42.

The first operation wire 41 may be a wire configured to operate the knife 2. The first operation wire 41 may include a coil shaft 44 and a tube 45. A distal end of the first operation wire 41 may be connected to the connecting member 22 of the knife 2, and a proximal end of the first operation wire 41 may be connected to a slider 52 of the operation unit 5. Further, the first operation wire 41 may be another aspect as long as it is a hollow shaft.

The coil shaft 44 may be a hollow coil wire formed of a metal. The coil shaft 44 may be formed of a material such as stainless steel or the like. The third pipeline 43 can be formed in the coil shaft 44. The third pipeline 43 can be connected to a proximal end of the second pipeline 24. The first pipeline 23, the second pipeline 24 and the third pipeline 43 can form a water supply flow path WR. A fluid supplied from a liquid supply port 54 can pass through the water supply flow path WR (the third pipeline 43, the second pipeline 24, and the first pipeline 23) and can be discharged from the distal end opening 23a.

The tube 45 may be a tube provided on an outer circumferential portion of the coil shaft 44, for example, a heat shrinkable tube. As the outer circumferential portion of the coil shaft 44 is covered with the tube 45, the liquid does not leak from the third pipeline 43.

The second operation wire 42 may be a wire configured to operate the sharp member 3. A distal end of the second operation wire 42 may be connected to the flange 33 of the sharp member 3, and a proximal end of the second operation wire 42 may be connected to a lever 55 of the operation unit 5.

As shown in FIG. 1 and FIG. 2, the operation unit 5 may include an operation unit main body 51, the slider 52, a feeding connector 53, the liquid supply port 54, and the lever 55.

A distal end portion of the operation unit main body 51 may be connected to the proximal end 1b of the sheath 1. The operation unit main body 51 may include an internal space through which the first operation wire 41 and the second operation wire 42 can be inserted. The first operation wire 41 can pass through the internal space 19 of the tube 10 and the internal space of the operation unit main body 51 and can extend to the slider 52. The second operation wire 42 can pass through the internal space 19 of the tube and the internal space of the operation unit main body 51 and can extend to the lever 55.

The slider 52 (first slider) may be attached to the operation unit main body 51 so as to be movable in the longitudinal direction A. A proximal end of the first operation wire 41 may be attached to the slider 52. As the operator advances and retracts the slider 52 relative to the operation unit main body 51, the first operation wire 41 and the knife 2 advance and retract.

The feeding connector 53 may be fixed to the slider 52. The feeding connector 53 can be connected to a high frequency power source device (not shown) and be connected to a proximal end portion of the first operation wire 41 via a conductive wire. The feeding connector 53 can supply the high frequency current supplied from the high frequency power source device to the knife 2 via the first operation wire 41. Further, the feeding connector 53 may be fixed to the operation unit main body 51 instead of the slider 52.

The liquid supply port 54 may be provided on the slider 52. The liquid supply port 54 may be connected to a proximal end of the third pipeline 43 via a pipeline formed in the slider 52. The liquid supplied from the liquid supply port 54 can pass through the water supply flow path WR (the third pipeline 43, the second pipeline 24, and the first pipeline 23) and can be discharged from the distal end opening 23a. In an example, the liquid supply port 54 may be provided in the operation unit main body 51 instead of the slider 52.

The lever 55 (second slider) may be attached to the slider 52 to be movable in the longitudinal direction A. A proximal end of the second operation wire 42 may be attached to the lever 55. As the operator advances and retracts the lever 55 relative to the slider 52, the second operation wire 42 and the sharp member 3 advance and retract.

### [Variant (Treatment Tool 100B) of Treatment Tool 100]

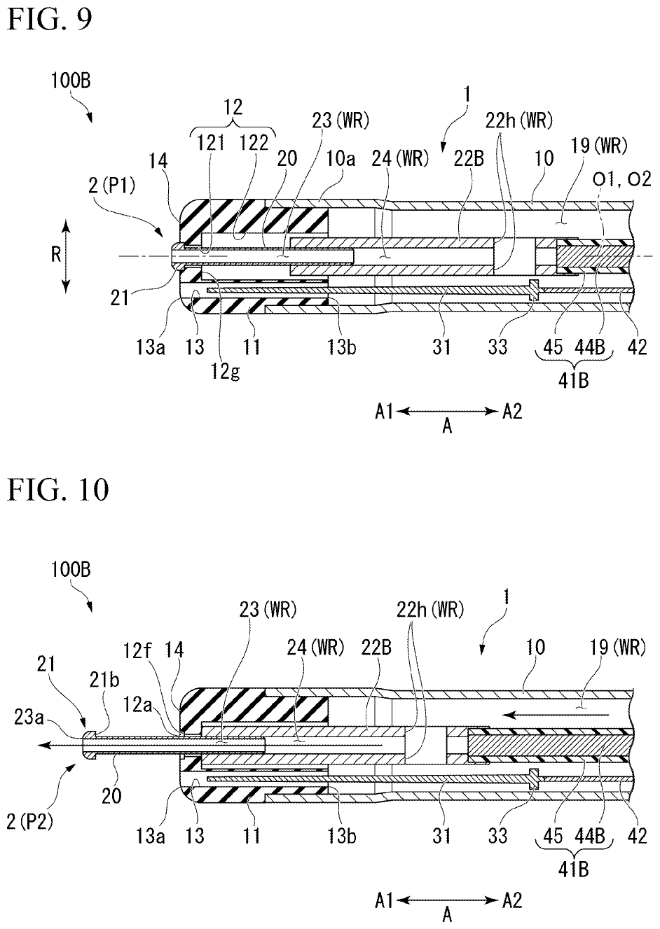

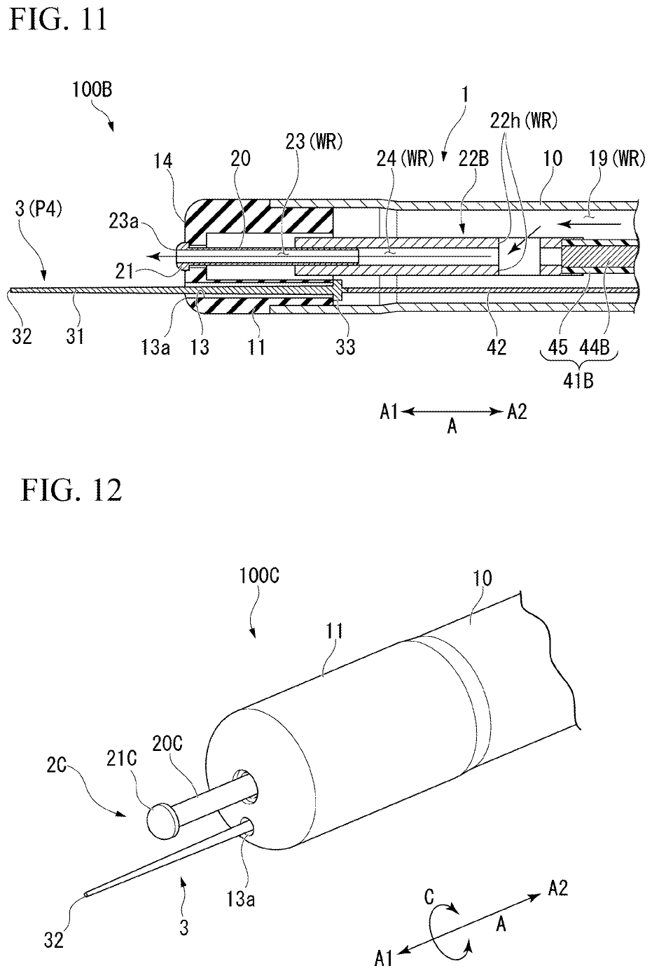

FIG. 9 to FIG. 11 are cross-sectional views showing a treatment tool 100B that is a variant of the treatment tool 100.

The treatment tool 100B is distinguished from the treatment tool 100 in the water supply flow path WR. The treatment tool 100B can include a sheath 1, a knife 2B, a sharp member 3, an operation wire 4B, and an operation unit 5. The knife 2B can include a knife main body 20, a flange 21, and a connecting member 22B.

The connecting member 22B may include a water supply port 22h, compared to the connecting member 22. The water supply port 22h may be a hole formed from the second pipeline 24 in the radial direction R, and can open in an outer circumferential surface of the connecting member 22B. The water supply ports 22h may be formed on both sides with the center axis O2 sandwiched in the longitudinal direction A of the knife 2B.

The operation wire 4B may be a wire inserted through an internal space (pipeline, lumen) 19 of the sheath 1. The operation wire 4B may include a first operation wire 41B, and a second operation wire 42.

The first operation wire 41B may be a wire configured to operate the knife 2B. The first operation wire 41B may include a coil shaft 44B, and a tube 45. A distal end of the first operation wire 41B can be connected to the connecting member 22B of the knife 2B, and a proximal end of the first operation wire 41B can be connected to the slider 52 of the operation unit 5.

The coil shaft 44B may be a coil shaft, may be a solid wire of a single wire, or a stranded wire formed of a metal, instead of the coil shaft. In an example, the third pipeline 43 is not formed in the coil shaft 44B. In such an example, the first operation wire 41B may not have the tube 45.

The liquid supply port 54 may be connected to a proximal end of the internal space 19 of the sheath 1 via a pipeline formed in the slider 52. The liquid supplied from the liquid supply port 54 can pass through the water supply flow path WR (the internal space 19 of the sheath 1, the water supply port 22h, the second pipeline 24, and the first pipeline 23) and can be discharged from the distal end opening 23a.

### [Method of Using Endoscope Treatment System 300]

Next, a technique using the endoscope treatment system 300 of the embodiment (a method of using the endoscope treatment system 300) will be described. Specifically, local injection treatment or incision or exfoliation treatment of a lesioned part in endoscopic therapy such as endoscopic submucosal dissection (ESD) or the like will be described.

As a preparing work, an operator can specify a lesioned part. Specifically, the operator can insert the insertion part 202 of the endoscope 200 into the digestive canal (for example, the gullet, the stomach, the duodenum, the large intestine), and specify a lesioned part while observing an image obtained by the imaging part 203 of the endoscope.

### <Insertion>

The operator can insert the treatment tool 100 into the channel 206, and cause the distal end 1a of the sheath 1 to protrude from the distal end opening portion 206a of the insertion part 202.

### <Marking>

As shown in FIG. 4, the operator can retract the slider 52 of the operation unit 5 relative to the operation unit main body 51 and dispose the distal end of the knife 2 at the first position P1. A biological tissue around the lesioned part can be cauterized and marked using the distal end portion of the flange 21 protruding from the distal end surface 14 of the distal end member 11.

### <Puncture>

As shown in FIG. 7, the operator can advance the lever 55 of the operation unit relative to the slider 52 and dispose the distal end of the sharp member 3 at the distal end position P4. The operator can puncture a place of the lesioned part into which a liquid for local injection (local injection liquid) can be injected using the sharp member 3 and passes therethrough.

### <Local Injection>

As shown in FIG. 5, the operator can advance the slider 52 of the operation unit relative to the operation unit main body 51 and dispose the distal end of the knife 2 at the second position P2. The operator can supply a liquid (local injection liquid) from the liquid supply port 54 in a state in which the distal end opening 23a of the distal end of the knife 2 is put into the submucosal layer from a hole punctured and formed by the sharp member 3. The liquid (local injection liquid) can be discharged from the distal end opening 23a.

### <Incision/Exfoliation>

Next, the operator can perform an incision or exfoliation treatment. The operator can advance the knife 2, move the flange 21 in a state in which the high frequency current is applied, and incise a mucous membrane of the lesioned part. In addition, the operator can advance the knife 2 and exfoliate the submucosal layer of the incised lesioned part while lifting the mucous membrane of the incised lesioned part and exposing the submucosal layer in a state in which the high frequency current is applied.

The operator can continue the above-mentioned operation (treatment) as necessary and finally incise the lesioned part, thereby terminating the ESD technique.

According to the treatment tool 100 and the treatment tool 100B of the embodiment, incision or exfoliation treatment and local injection treatment can be appropriately performed, with easier operator handling of the treatment tool 100 and 100B.

Hereinabove, while the first embodiment has been described in detail with reference to the accompanying drawings, the specific configuration is not limited to the embodiment and may include design changes or the like without departing from the scope of the present disclosure. In addition, the components shown in the above-mentioned embodiment and variants thereof can be configured by appropriately combining them.

### Second Embodiment

A treatment tool 100C according to a second embodiment will be described with reference to FIG. 12 to FIG. 16. In the following description, the common configurations described above are designated by the same reference signs and overlapping descriptions will be omitted.

FIG. 12 is a perspective view of a distal end portion of the treatment tool 100C. The treatment tool (treatment tool for an endoscope) 100C may constitute an endoscope treatment system together with the endoscope 200, like the treatment tool 100 of the first embodiment. The treatment tool 100C may include a sheath 1, a knife 2C, a sharp member 3, an operation wire 4B, and an operation unit 5.

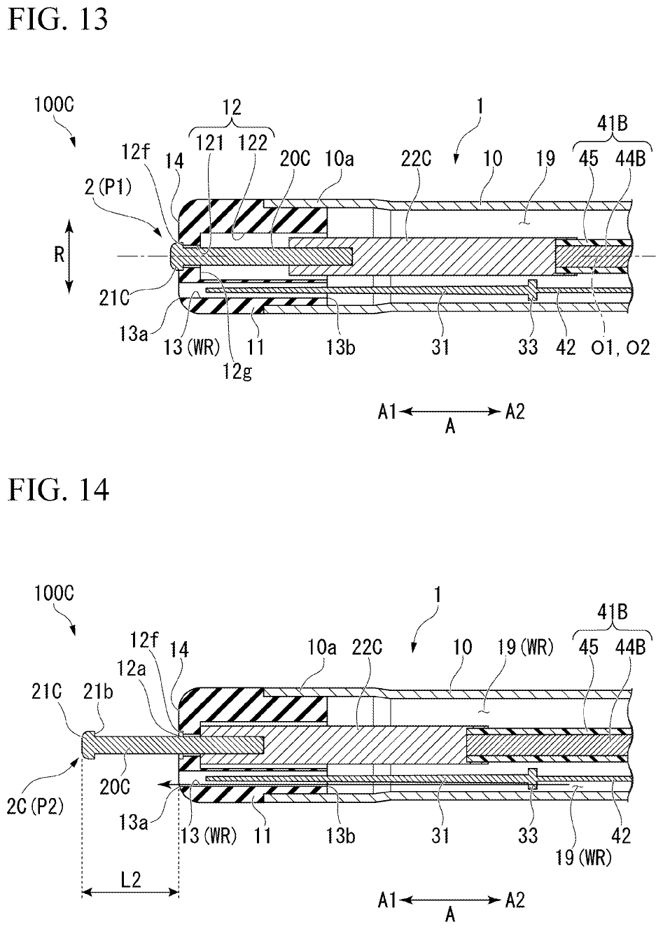

FIG. 13 to FIG. 15 are cross-sectional views of the distal end portion of the treatment tool 100C.

The knife (electrode) 2C may be a round rod-shaped (or a substantially round rod-shaped) member formed of a metal. Thus, “round rod shape” can include a shape close to a round rod shape, in addition to a strict round rod shape. The knife 2C may be formed of a material such as stainless steel or the like. The knife 2C can have a conductivity and can be energized with high frequency current. The knife 2C may include a knife main body 20C, a flange 21C, and a connecting member 22C.

The knife 2C can be inserted through the first through-hole 12 of the distal end member 11 of the sheath 1 in the longitudinal direction A, and can freely protrude from and be inserted into the first opening 12a at the distal end side A1. Further, the knife 2C may be fixed not to advance and retract while protruding from the first opening 12a to the distal end side A1.

The center axis O2 of the knife 2C in the longitudinal direction A may match (or substantially match) the center axis O1 of the sheath 1 in the longitudinal direction A. Thus, “match” includes not only an aspect of exactly matching but also an aspect of almost matching.

The knife main body 20C may be a round rod-shaped member formed of a metal. An outer diameter of the knife main body 20C may be smaller than an inner diameter of the first region 121. The knife main body 20C can move the first region 121 forward and rearward in the longitudinal direction A.

The flange 21C (distal end diameter expansion part) may be a disk-shaped conductive member provided on the distal end of the knife main body 20. Like the first embodiment, when seen in a front view viewed in the longitudinal direction A, an outer circumference of the flange 21C can be formed in a circular shape concentric with an outer circumference of the knife main body 20C. A length of the flange 21C in the radial direction R perpendicular to the longitudinal direction A may be greater than a length of the knife main body 20C in the radial direction R. A planar proximal end surface 21b may be formed on the proximal end side A2 of the flange 21C. The flange 21C is not limited to a disk shape and may have a triangular shape or a hook shape.

As shown in FIG. 13, when the knife 2C is retracted against the sheath 1, the proximal end surface 21b of the knife 2C can come into contact with the flange storage part 12f of the distal end member 11. As the proximal end surface 21b of the knife 2C comes into contact with the flange storage part 12f of the distal end member 11, the distal end of the knife 2C can be positioned at the first position P1 that is a position closest to the proximal end side A2.

In an example, the knife main body 20C and the flange 21C do not have the first pipeline 23 extending in the longitudinal direction A.

The connecting member 22C may be a columnar member formed of a metal. The “columnar shape” can also include a shape close to a columnar shape, in addition to a strict columnar shape. The connecting member 22C can connect the knife main body 20C and the first operation wire 41B. An outer diameter of the connecting member 22C may be greater than an inner diameter of the first region 121 and smaller than an inner diameter of the second region 122. In an example, the connecting member 22C cannot be inserted through the first region 121 in the longitudinal direction A, and can move the second region 122 forward and rearward in the longitudinal direction A.

As shown in FIG. 14, when the knife 2C advances against the sheath 1, the distal end of the connecting member 22C can come into contact or make contact with the step difference 12g of the distal end member 11. As the distal end of the connecting member 22C comes into contact with the step difference 12g of the distal end member 11, the distal end of the knife 2C can be positioned at the second position P2 that is a position closest to the distal end side A1.

In an example, the connecting member 22C does not have the second pipeline 24 extending in the longitudinal direction A.

The first operation wire 41B can be attached to a proximal end of the connecting member 22C. A high frequency current can be supplied to the knife 2C from the first operation wire 41B connected to the operation unit 5. When the high frequency current is supplied to the knife 2C via the first operation wire 41B, the knife main body 20C and the flange 21C can function as an active electrode configured to output a high frequency current to a biological tissue.

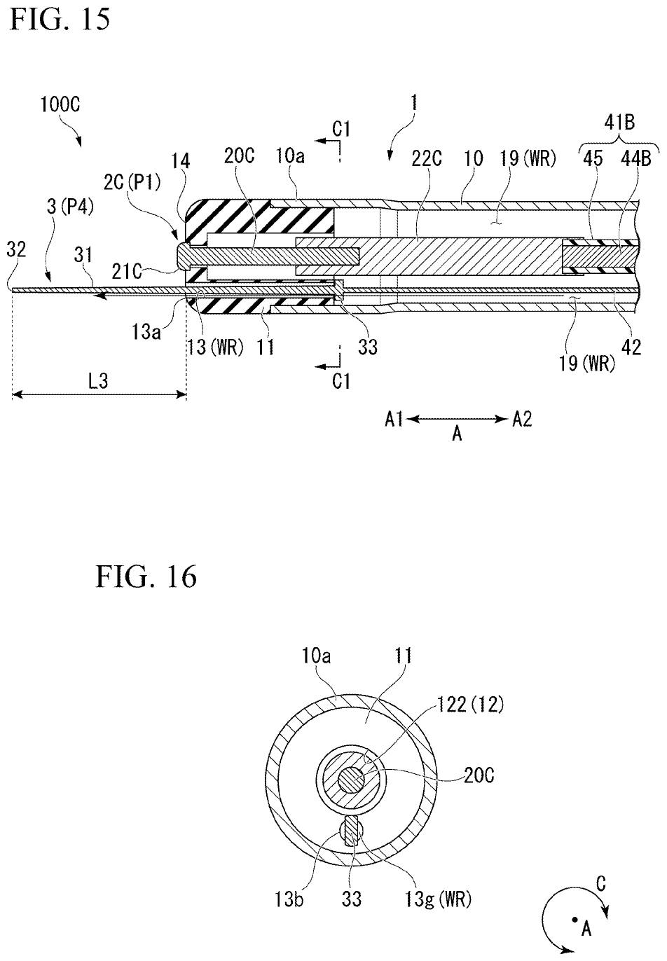

FIG. 16 is a cross-sectional view along line C1-C1 shown in FIG. 15.

The flange (diameter expansion part) 33 can be provided on a proximal end of the main body portion 31. An outer diameter of at least a part of the flange 33 may be greater than an inner diameter of the second through-hole 13. In an example, the flange 33 cannot be inserted through the second through-hole 13.

As shown in FIG. 15, when the sharp member 3 advances against the sheath 1, the flange 33 of the sharp member 3 can come into contact with or make contact with the proximal end opening 13b of the second through-hole 13 of the distal end member 11. As the flange 33 of the sharp member 3 comes into contact with the proximal end opening 13b of the second through-hole 13, the distal end of the sharp member 3 (the distal end of the sharp portion 32) can be positioned at the distal end position P4 that is a position closest to the distal end side A1.

As shown in FIG. 16, when seen in a direction along the longitudinal direction A, the flange 33 may be formed in a rectangular shape. When the flange 33 comes into contact with the proximal end opening 13b of the second through-hole 13, a gap 13g is provided between the flange 33 of the sharp member 3 and the proximal end opening 13b. The gap 13g can be a part of the water supply flow path WR through which the liquid flows. Further, the flange 33 may also have an oval shape or an elongated circular shape when seen in a direction along the longitudinal direction A, for example, as long as it is a shape formed by the gap 13g when coming in contact with the proximal end opening 13b.

The liquid supply port 54 can be connected to a proximal end of the internal space 19 of the sheath 1 via the pipeline formed in the slider 52. The liquid supplied from the liquid supply port 54 can pass through the water supply flow path WR (the internal space 19 of the sheath 1, the gap 13g and the second through-hole 13) and can be discharged from the second opening 13a.

The protrusion amount L3 of the sharp member 3 (a maximum protrusion amount of the sharp member 3, see FIG. 15) protruding from the second opening 13a when the distal end of the sharp portion 32 is disposed at the distal end position P4 may be greater than the protrusion amount L2 of the knife 2C (a maximum protrusion amount of the knife 2C, see FIG. 14) protruding from the first opening 12a when the distal end of the knife 2C is disposed at the second position P2.

Further, the protrusion amount L3 of the sharp member 3 protruding from the second opening 13a when the distal end of the sharp portion 32 is disposed at the distal end position P4 may be smaller than the protrusion amount L2 of the knife 2C maximally protruding from the first opening 12a when the distal end of the knife 2C is disposed at the second position P2.

### [Variant (Treatment Tool 100D) of Treatment Tool 100C]

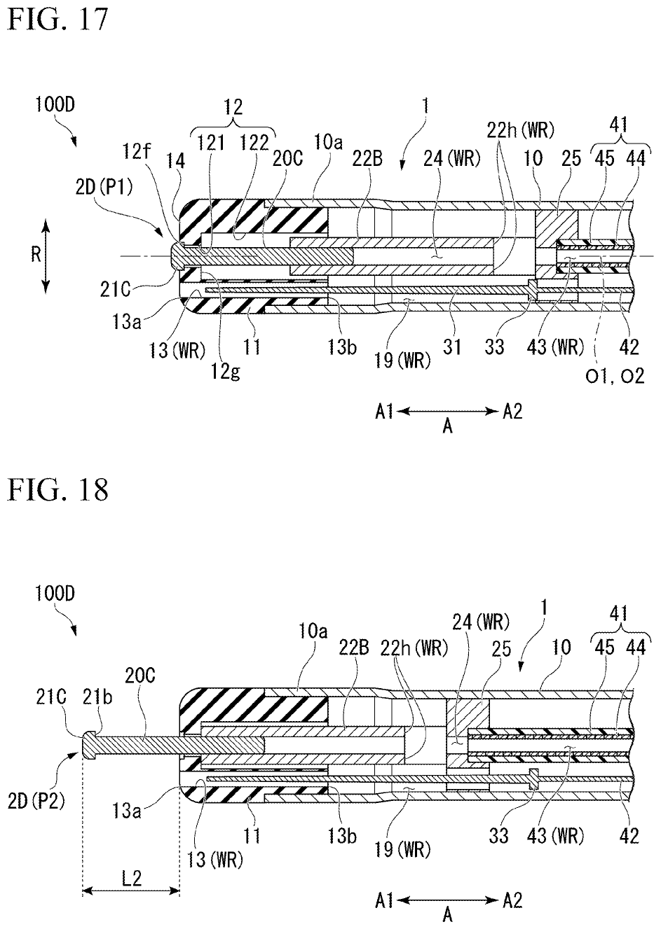

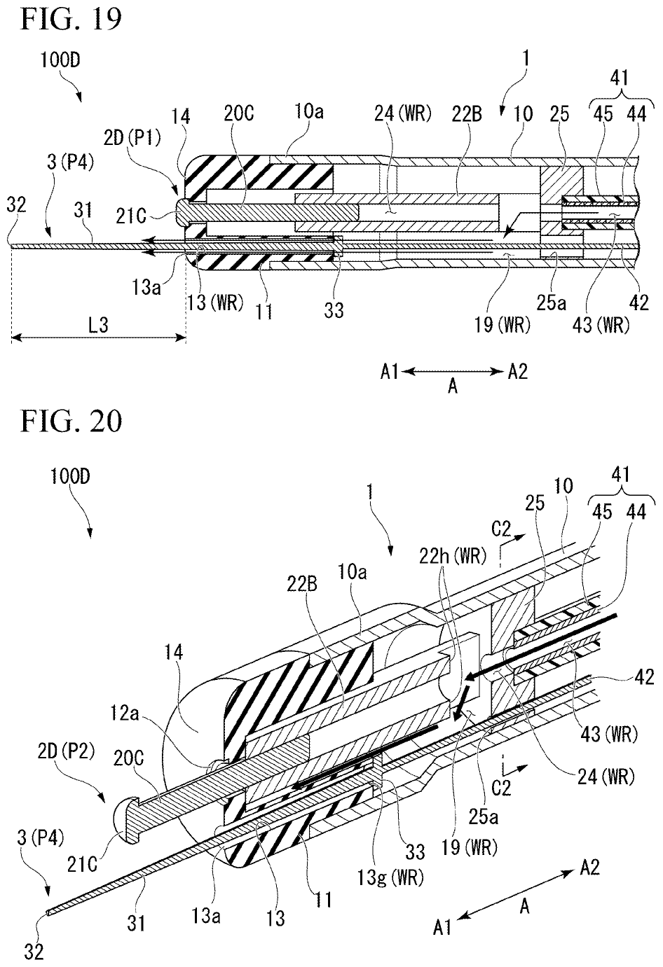

FIG. 17 to FIG. 19 are cross-sectional views showing a treatment tool 100D that is a variant of the treatment tool 100C. The treatment tool 100D is distinguished from the treatment tool 100C in the water supply flow path WR. The treatment tool 100D may include a sheath 1, a knife 2D, a sharp member 3, an operation wire 4, and an operation unit 5. The knife 2D has a knife main body 20C, a flange 21C, a connecting member 22B, and a counterflow prevention member 25.

FIG. 20 is a perspective cross-sectional view showing the counterflow prevention member 25.

The counterflow prevention member 25 may be a disk-shaped member and may be provided or located closer to the proximal end side A2 than the water supply port 22h of the connecting member 22B. The counterflow prevention member 25 can prevent the liquid discharged to the outside of the connecting member 22B from the water supply port 22h from counter flowing to the proximal end side A2. The counterflow prevention member 25 is not limited to the member fixed to the connecting member 22B, and may be a member that slides along the connecting member 22B, for example, an O-ring formed of urethane rubber or the like. In addition, the counterflow prevention member 25 is not limited to the member attached to the outer circumferential surface of the connecting member 22B and may be a member attached to the inner circumferential surface of the tube 10.

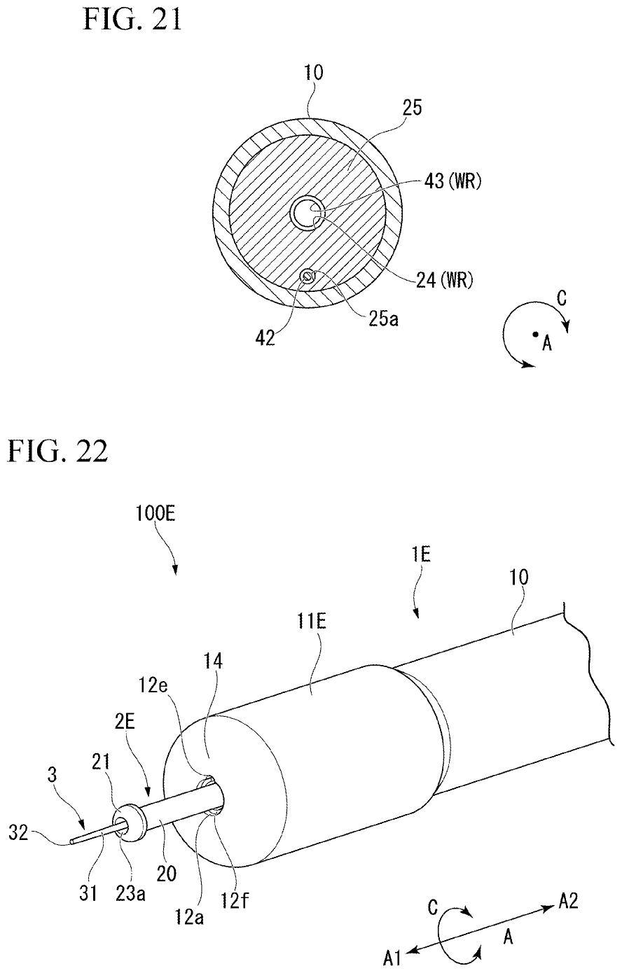

FIG. 21 is a cross-sectional view along line C2-C2 shown in FIG. 20.

The counterflow prevention member 25 may be a member configured to cover the internal space 19 when seen in a direction along the longitudinal direction A. The counterflow prevention member 25 may include a through-hole 25a through which the second operation wire 42 can be inserted.

The liquid supply port 54 may be connected to the proximal end of the third pipeline 43 of the sheath 1 via the pipeline formed in the slider 52. The liquid supplied from the liquid supply port 54 can pass through the water supply flow path WR (the third pipeline 43, the second pipeline 24, the water supply port 22h, the internal space 19 of the sheath 1, and the second through-hole 13) and can be discharged from the second opening 13a.

According to the treatment tool 100C and the treatment tool 100D of the embodiment, incision or exfoliation treatment and local injection treatment can be appropriately performed, and the treatment tool 100C and 100D can be more easily handled by a user or operator.

Hereinabove, while the second embodiment has been described in detail with reference to the accompanying drawings, the specific configuration is not limited to the embodiment and may include design changes or the like without departing from the scope of the present disclosure. In addition, the components shown in the above-mentioned embodiment and variant can be configured by appropriately combining them.

### Third Embodiment

A treatment tool 100E according to a third embodiment will be described with reference to FIG. 22 to FIG. 26. In the following description, the common configurations described above are designated by the same reference signs and overlapping descriptions will be omitted.

FIG. 22 is a perspective view of a distal end portion of the treatment tool 100E.

The treatment tool (treatment tool for an endoscope) 100E may constitute an endoscope treatment system together with the endoscope 200, like the treatment tool 100 of the first embodiment. The treatment tool 100E may include a sheath 1E, a knife 2E, a sharp member 3, an operation wire 4, and an operation unit 5.

The sheath 1E may be an elongated tubular member extending from the distal end 1a to the proximal end 1b. The sheath 1E can include an outer diameter that can be inserted into the channel 206 of the endoscope 200 and can move forward and rearward in the channel 206. The sheath 1E may include a tube 10 extending in the longitudinal direction A, and a distal end member 11E provided on a distal end of the tube 10. Further, the tube 10 and the distal end member 11E of the sheath 1E may be formed integrally with each other.

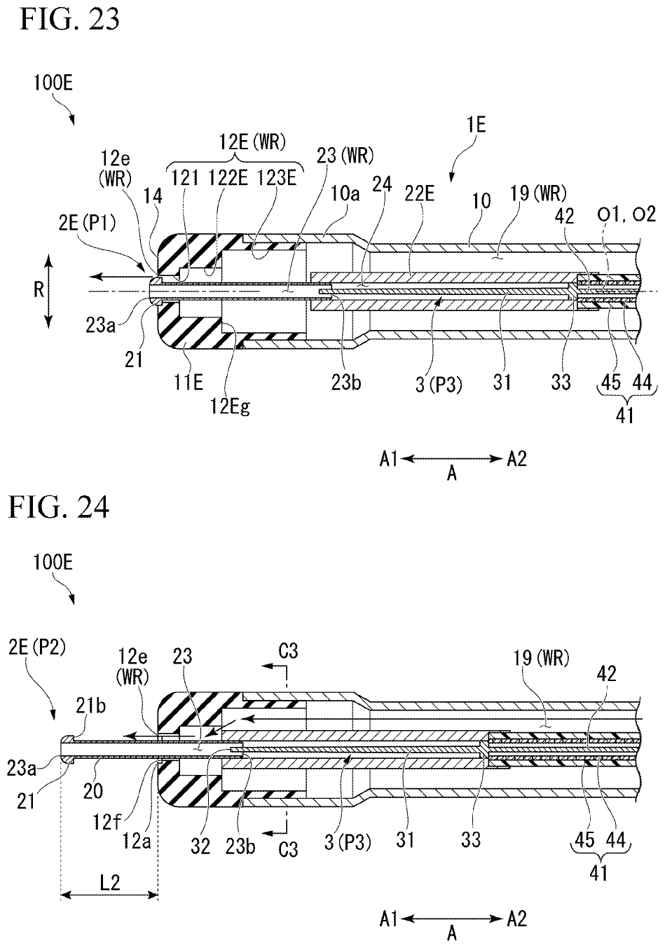

FIG. 23 to FIG. 25 are cross-sectional views of the distal end portion of the treatment tool 100E.

The distal end member 11E may be formed in a cylindrical shape or a substantially cylindrical shape. Thus, the “cylindrical shape” can also include a shape close to a cylindrical shape, in addition to a strict cylindrical shape. The distal end member 11E may be formed of an insulating material such as a resin or the like. A first through-hole 12E may be formed in the distal end member 11E.

The first through-hole 12E may be a hole provided in the distal end member 11E and passing through the distal end member 11E in the longitudinal direction A. The distal end of the first through-hole 12E may be in communication with or next to, or the like, the first opening 12a formed in the distal end surface 14 of the distal end member 11E. The proximal end of the first through-hole 12E may be in communication with the internal space 19 of the tube 10. The knife 2E may be inserted through the first through-hole 12E.

The first through-hole 12E may include a first region 121 on the distal end side A1, a second region 122E, and/or a third region 123E on the proximal end side A2. The second region 122E may be a part of the through-hole passing through in the longitudinal direction A, and a cross section perpendicular to the longitudinal direction A may have a circular shape. The third region 123E may be a part of the through-hole passing through in the longitudinal direction A, and a cross section perpendicular to the longitudinal direction A may have a circular shape. The first region 121 may be in communication with, next to, connected to, proximate to, abutting, or the like, the second region 122E. The second region 122E may be in communication with, next to, connected to, proximate to, abutting, or the like, the third region 123E. An inner diameter of the first region 121 may be smaller than that of the second region 122E. The inner diameter of the second region 122E may be smaller than that of the third region 123E. A step difference 12Eg may be formed between the second region 122E and the third region 123E.

A flange storage part 12f configured to accommodate the flange 21 of the knife 2E, and a water supply groove 12e may be formed in the first opening 12a of the first through-hole 12E of the distal end member 11E. In an example, even when the flange 21 is stored in the flange storage part 12f, the water supply groove 12e may not not closed by the flange 21, and the liquid supplied to the first through-hole 12 can be discharged from the water supply groove 12e.

The knife (electrode) 2E may be a round rod-shaped member formed of a metal. The “round rod shape” also includes a shape close to a round rod shape, in addition to a strict round rod shape. The knife 2E may be formed of a material such as stainless steel or the like. The knife 2E may have a conductivity and can be energized with a high frequency current. The knife 2E may include a knife main body 20, a flange 21, and a connecting member 22E.

As shown in FIG. 23, when the knife 2E is retracted against the sheath 1E, the proximal end surface 21b of the knife 2E may come into contact with the flange storage part 12f of the distal end member 11E. As the proximal end surface 21b of the knife 2E comes into contact with the flange storage part 12f of the distal end member 11E, the distal end of the knife 2E can be positioned at the first position P1 that is a position closest to the proximal end side A2.

FIG. 26 is a cross-sectional view along line C3-C3 shown in FIG. 24.

The connecting member 22E may be a member formed of a metal, and may be formed in a shape in which both sides of the cylindrical shape in the radial direction R are cut off. The connecting member 22E may connect the knife main body 20 and the first operation wire 41. The connecting member 22E may have a maximum width greater than an inner diameter of the first region 121 in the radial direction R. The connecting member 22E may have a maximum width greater than the inner diameter of the second region 122E in the radial direction R. At least a part of the connecting member 22E may have a width smaller than the inner diameter of the second region 122E in the radial direction R. Specifically, the connecting member 22E may be formed in a shape in which a part of a circle is cut off when seen in a direction in the longitudinal direction A. The maximum width of the connecting member 22E in the radial direction R may be smaller than the inner diameter of the third region 123E. In such an example, the connecting member 22 cannot be inserted through the first region 121 and the second region 122E in the longitudinal direction A, and can move the third region 123E forward and rearward in the longitudinal direction A.

As shown in FIG. 24, when the knife 2E can advance against the sheath 1E, the distal end of the connecting member 22E may come into contact with the step difference 12Eg of the distal end member 11E. As the distal end of the connecting member 22E comes into contact with the step difference 12Eg of the distal end member 11E, the distal end of the knife 2E can be positioned at the second position P2 that is a position closest to the distal end side A1.

The connecting member 22E has the second pipeline 24 extending in the longitudinal direction A. The second pipeline 24 may be in communication with the third pipeline 43 formed in the first pipeline 23 and the first operation wire 41. In the embodiment, the first pipeline 23, the second pipeline 24 and the third pipeline 43 are not used as the water supply flow path WR and are used as a pipeline through which the sharp member 3 advances and retracts.

The first operation wire 41 may be attached to the proximal end of the connecting member 22E. A high frequency current can be supplied to the knife 2E via the first operation wire 41 connected to the operation unit 5. When the high frequency current is supplied to the knife 2E from the first operation wire 41, the knife main body and the flange 21 can function as an active electrode configured to output a high frequency current to a biological tissue.

The sharp member 3 may be inserted through the first pipeline 23, the second pipeline 24 and the third pipeline 43 in the longitudinal direction A, and can freely protrude from and be inserted into the distal end opening 23a on the distal end side A1.

The flange (diameter expansion part) 33 can be provided on the proximal end of the main body portion 31. An outer diameter of the flange 33 may be smaller than an inner diameter of the second pipeline 24. An outer diameter of at least a part of the flange 33 may be greater than an inner diameter of a proximal end opening 23b of the first pipeline 23 and an inner diameter of a distal end opening 43a of the third pipeline 43. In an example, the flange 33 can advance and retract through the second pipeline 24 and cannot be inserted through the first pipeline 23 and the third pipeline 43.

As shown in FIG. 24, when the sharp member 3 is retracted against the sheath 1E and the knife main body 20, the flange 33 of the sharp member 3 may come into contact with the distal end opening 43a of the third pipeline 43 of the first operation wire 41. As the flange 33 of the sharp member 3 comes into contact with the distal end opening 43a of the third pipeline 43, the distal end of the sharp member 3 can be positioned at a proximal end position P3 that is a position closest to the proximal end side A2.

When the distal end of the sharp member 3 is disposed at the proximal end position P3, the sharp portion 32 of the sharp member 3 can be stored in the second pipeline 24.

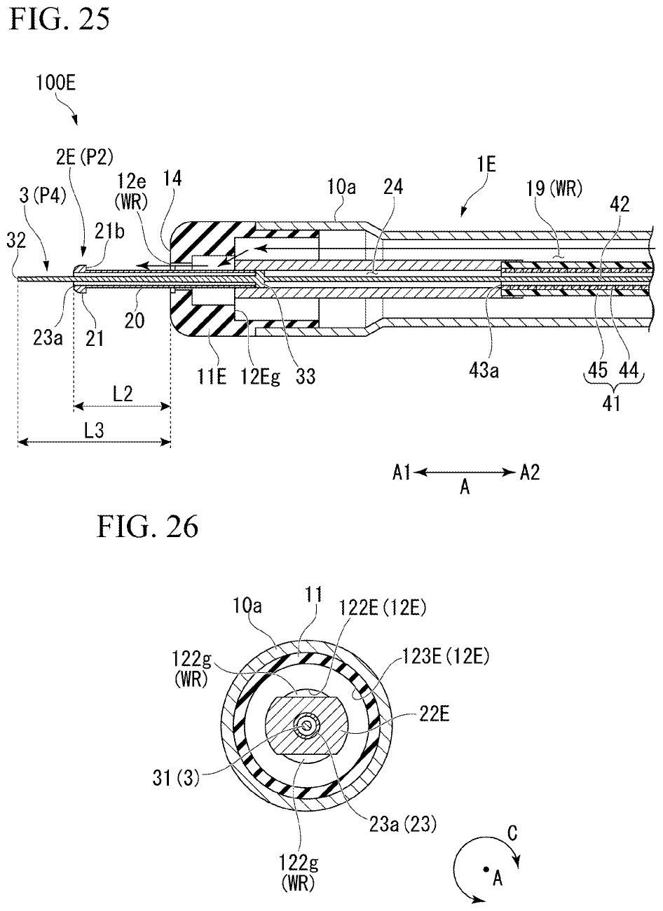

As shown in FIG. 25, when the sharp member 3 advances against the sheath 1E and the knife main body 20, the flange 33 of the sharp member 3 may come into contact with the proximal end opening 23b of the first pipeline 23 of the knife main body 20.

As the flange 33 of the sharp member 3 comes into contact with the proximal end opening 23b of the first pipeline 23, the distal end of the sharp member 3 (the distal end of the sharp portion 32) can be positioned at the distal end position P4 that is a position closest to the distal end side A1.

As shown in FIG. 26, the connecting member 22E may be formed in a shape in which a part of a circle is cut off when seen in a direction along the longitudinal direction A. When the connecting member 22E comes into contact with the second region 122E, a gap 122g may be formed between the connecting member 22E and the second region 122E. In such an example, the gap 122g is a part of the water supply flow path WR through which a liquid flows.

The liquid supply port 54 may be connected to the proximal end of the internal space 19 of the sheath 1E via the pipeline formed in the slider 52. In such an example, the liquid supplied from the liquid supply port 54 can pass through the water supply flow path WR (the internal space 19 of the sheath 1E, the first through-hole 12, the gap 122g, and the water supply groove 12e) and is discharged from the first opening 12a.

The protrusion amount L3 (a maximum protrusion amount of the sharp member 3, see FIG. 25) of the sharp member 3 protruding from the first opening 12a when the distal end of the sharp portion 32 is disposed at the distal end position P4 may be greater than the protrusion amount L2 (a maximum protrusion amount of the knife 2E, see FIG. 24) of the knife 2E protruding from the first opening 12a when the distal end of the knife 2E is disposed at the second position P2.

A maximum protrusion amount of the sharp member 3 protruding from a first opening 23a when the distal end of the sharp portion 32 is disposed at the distal end position P4 may be smaller than the protrusion amount L2. Further, the maximum protrusion amount of the sharp member 3 protruding from the first opening 23a when the distal end of the sharp portion 32 is disposed at the distal end position P4 may be greater than the protrusion amount L2.

### [Variant (Treatment Tool 100F) of Treatment Tool 100E]

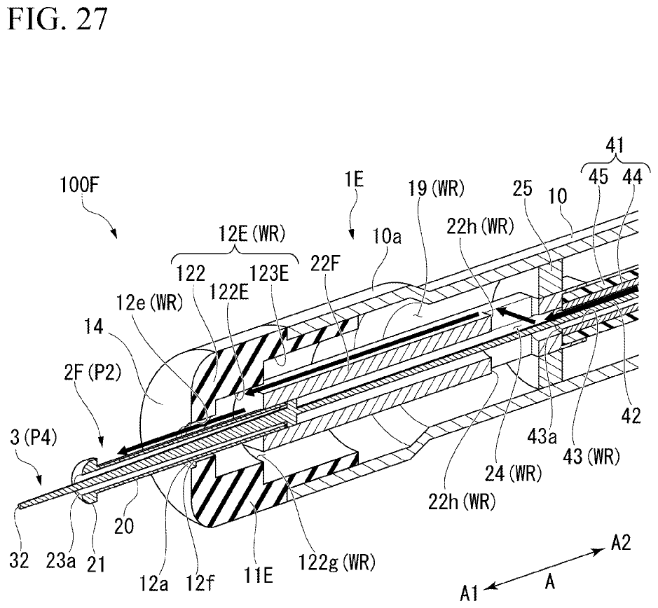

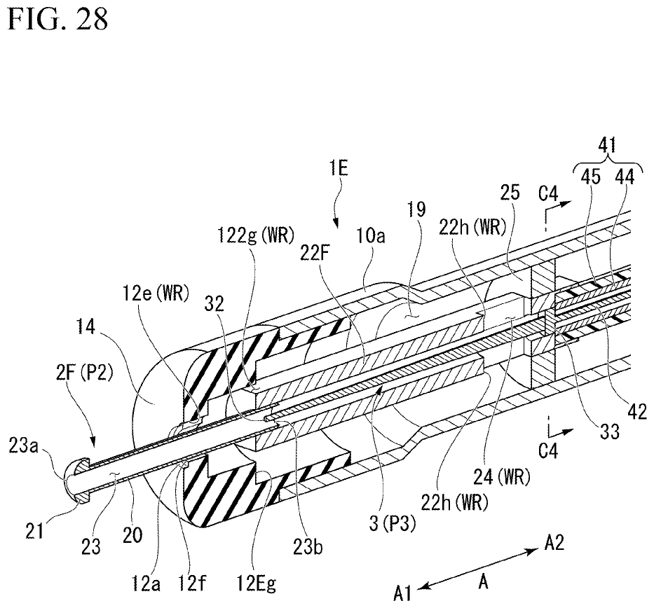

FIG. 27 to FIG. 28 are perspective cross-sectional views showing a treatment tool 100F that is a variant of the treatment tool 100E. The treatment tool 100F is distinguished from the treatment tool 100E in the water supply flow path WR. The treatment tool 100F includes a sheath 1E, a knife 2F, a sharp member 3, an operation wire 4, and an operation unit 5. The knife 2F has a knife main body 20, a flange 21, a connecting member 22F, and a counterflow prevention member 25.

The connecting member 22F may further include a water supply port 22h compared to the connecting member 22E. The water supply port 22h may be a hole formed from the second pipeline 24 in the radial direction R, and may open in an outer circumferential surface of the connecting member 22F. The water supply ports 22h may be formed at both sides of the knife 2F with the center axis O2 sandwiched or located therebetween in the longitudinal direction A.

The counterflow prevention member 25 may be a disk-shaped (or substantially disk-shaped) member provided or located closer to the proximal end side A2 than the water supply port 22h of the connecting member 22F. The counterflow prevention member 25 can prevent the liquid discharged to the outside of the connecting member 22F from the water supply port 22h from counter flowing to the proximal end side A2.

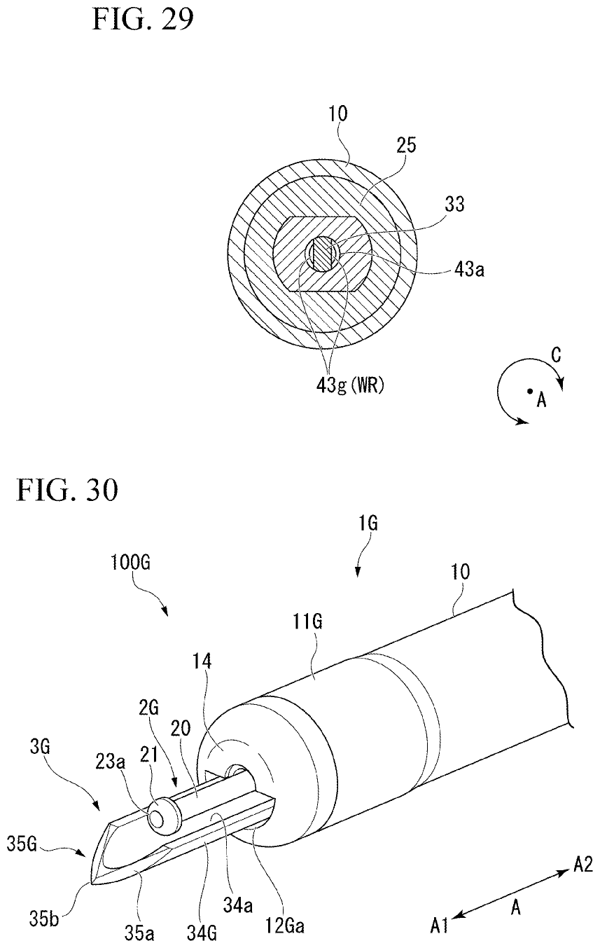

FIG. 29 is a cross-sectional view along line C4-C4 shown in FIG. 28.

The counterflow prevention member 25 may be a member configured to cover the internal space 19 when seen in a direction along the longitudinal direction A. When the flange 33 of the sharp member 3 comes into contact with the distal end opening 43a of the third pipeline 43, a gap 43g may be formed between the flange 33 and the distal end opening 43a. The gap 43g may be a part of the water supply flow path WR through which the liquid flows.

The liquid supply port 54 may be connected to a proximal end of the third pipeline 43 of the sheath 1E via the pipeline formed in the slider 52. The liquid supplied from the liquid supply port 54 can pass through the water supply flow path WR (the third pipeline 43, the gap 43g, the second pipeline 24, the water supply port 22h, the internal space 19 of the sheath 1E, the first through-hole 12, the gap 122g, and the water supply groove 12e) and can be discharged from the first opening 12a.

According to the treatment tool 100E and the treatment tool 100F of the embodiment, incision or exfoliation treatment and local injection treatment can be appropriately performed, and the treatment tool 100E and 100F may be more easily handled by a user or operator.

Hereinabove, while the third embodiment has been described in detail with reference to the accompanying drawings, the specific configuration is not limited to the embodiment and may include design changes or the like without departing from the scope of the present disclosure. In addition, the components shown in the above-mentioned embodiment and any variants can be configured by appropriately combining them.)

### Fourth Embodiment

A treatment tool 100G according to a fourth embodiment will be described with reference to FIG. 30 to FIG. 34. In the following description, the common configurations described above are designated by the same reference signs and overlapping descriptions will be omitted.

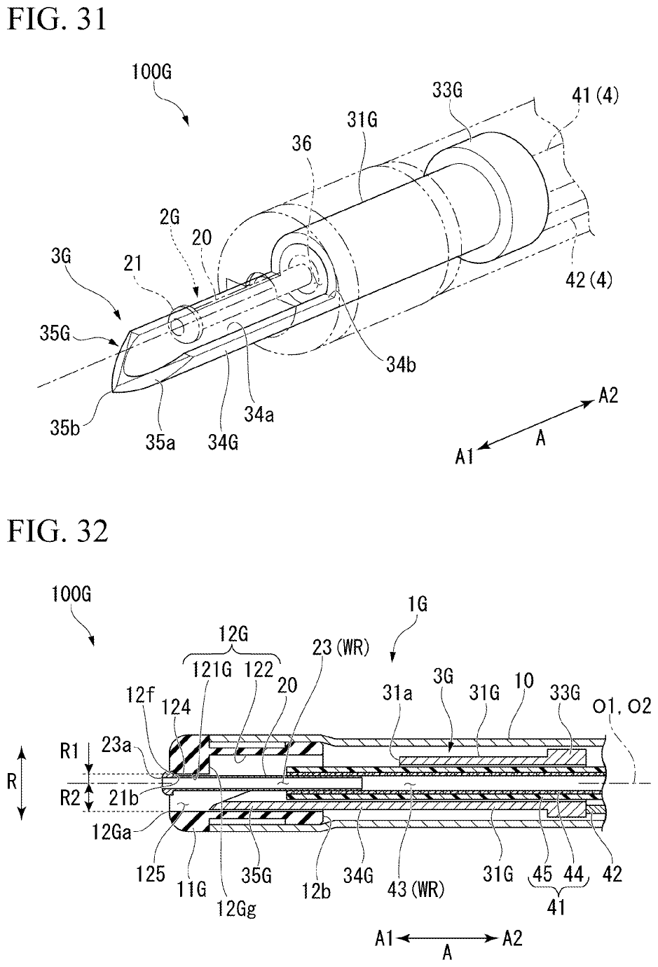

FIG. 30 and FIG. 31 are perspective views of a distal end portion of a treatment tool 100G.

The treatment tool (treatment tool for an endoscope) 100G may constitute an endoscope treatment system together with the endoscope 200, like the treatment tool 100 of the first embodiment. The treatment tool 100G may include a sheath 1G, a knife 2G, a sharp member (needle) 3G, an operation wire 4, and an operation unit 5.

The sheath 1G may be an elongate tubular member from the distal end 1a to the proximal end 1b. The sheath 1G has an outer diameter that can be inserted into the channel 206 of the endoscope 200 and can advance and retract through the channel 206. The sheath 1G may include a tube 10 extending in the longitudinal direction A, and a distal end member 11G provided on a distal end of the tube 10. Further, the tube 10 and the distal end member 11G of the sheath 1G may be formed integrally with each other.

FIG. 32 to FIG. 34 are cross-sectional views of a distal end portion of the treatment tool 100G.

The distal end member 11G is formed in a cylindrical shape or a substantially cylindrical shape. Thus, the “cylindrical shape” also includes a shape close to a cylindrical shape, in addition to a strict cylindrical shape. The distal end member 11G may be formed of an insulating material such as a resin or the like. A first through-hole 12G may be formed in the distal end member 11G.

The first through-hole 12G may be a hole provided in the distal end member 11G and passing through the distal end member 11G in the longitudinal direction A. The distal end of the first through-hole 12G may be in communication with a first opening 12Ga formed in the distal end surface 14 of the distal end member 11G. A proximal end of the first through-hole 12G may be in communication with the internal space 19 of the tube 10. The knife 2G and the sharp member 3G may be inserted through the first through-hole 12G.

The first through-hole 12G may include a first region 121G on the distal end side A1, and a second region 122 on the proximal end side A2. The first region 121G may be a through-hole whose cross section perpendicular to the longitudinal direction A is formed by overlapping two concentric circles with different diameters. The second region 122 may be a part of the through-hole passing through in the longitudinal direction A, and a cross section perpendicular to the longitudinal direction A may have a circular shape. The first region 121G may be in communication with, connect to, abut, or be located next to the second region 122. An inner diameter of the first region 121G may be smaller than that of the second region 122. A step difference 12Gg may be formed between the first region 121G and the second region 122. Further, a center axis of the second region 122 may coincides with the center axis O2, and a center axis of the first region 121G may be eccentric with the center axis O2.

FIG. 35 is a cross-sectional view along line C5-C5 shown in FIG. 34.

As shown in FIG. 32, the first region 121G may be divided into a diameter reduction region 124 in which a length of the knife 2G from the center axis O2 in the longitudinal direction A is a length R1, and a diameter expansion region 125 in which a length from the center axis O2 is a length R2 (where length R2>length R1). In the embodiment, the diameter reduction region 124 and the diameter expansion region 125 may each have a semi-circular shape when seen in a direction along the longitudinal direction A.

The knife 2G may be a round rod-shaped member formed of a metal. The round rod shape” may also include a shape close to a round rod shape, in addition to a strict round rod shape. The knife 2G may be formed of a material such as stainless steel or the like. The knife 2G may have conductivity and can be energized with a high frequency current. The knife 2G may include a knife main body 20 and a flange 21. In an example, the knife 2G does not have a connecting member 22. The knife main body 20 may be directly connected to the first operation wire 41, and the first pipeline 23 is in communication with the third pipeline 43.

The center axis O2 of the knife 2G in the longitudinal direction A may match or substantially match the center axis O1 of the sheath 1G in the longitudinal direction A. In an example, “match” includes not only an aspect of exactly matching but also an aspect of almost matching.

The knife main body 20 can advance and retract in the first region 121G. A radius of the knife main body 20 may be smaller than the length R1. For this reason, the knife main body 20 can advance and retract through the diameter reduction region 124 in the first region 121G.

As shown in FIG. 32, when the knife 2G is retracted against the sheath 1G, the proximal end surface 21b of the knife 2G may come into contact with a flange storage part 12F (a first surface) of the distal end member 11G. As the proximal end surface 21b of the knife 2G comes into contact with the flange storage part 12f of the distal end member 11G, the distal end of the knife 2G can be positioned at the first position P1 that is a position closest to the proximal end side A2. In a state in which the proximal end surface 21b of the knife 2G is in contact with the flange storage part 12f of the distal end member 11G, the sharp member 3G can move forward and rearward with respect to the knife 2G in the longitudinal direction A. Further, the flange storage part 12f may be not formed in the first opening 12Ga. In this case, a distal end surface of the distal end member 11G may be a first surface, and comes into contact with the proximal end surface 21b of the knife 2G.

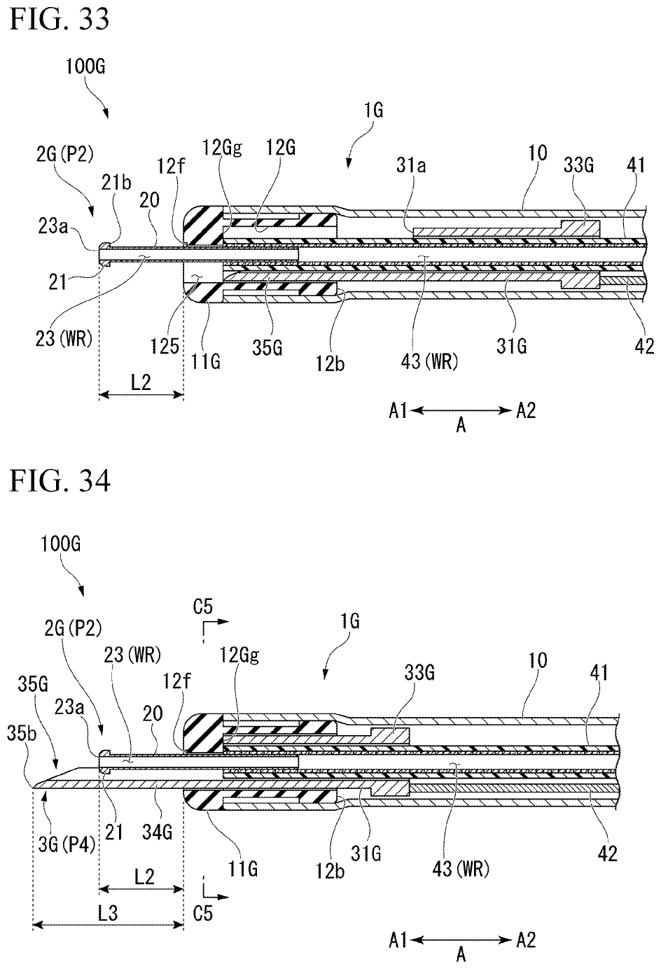

As shown in FIG. 33, when the knife 2G advances against the sheath 1G, a distal end of the first operation wire 41 can come into contact with a step difference (second surface) 12Gg of the distal end member 11G. As the distal end of the first operation wire 41 comes into contact with the step difference 12Gg of the distal end member 11G, the distal end of the knife 2G can be positioned at the second position P2 that is a position closest to the distal end side A1. In a state in which the connecting member 22C is in contact with the step difference 12Gg of the distal end member 11G, the sharp member 3G can move forward and rearward with respect to the knife 2G in the longitudinal direction A.

The sharp member (rod) 3G may be formed of a resin material, a metal material, a ceramic material, or the like. The sharp member 3G may include a main body portion 31G, a flange 33G, a distal end portion 34G, and a sharp portion 35G.

The sharp member 3G may be inserted through the first through-hole 12G of the distal end member 11G of the sheath 1G in the longitudinal direction A, and can freely protrude from and be inserted into the first opening 12Ga on the distal end side A1. The distal end portion 34G of the sharp member 3G can be provided to move forward and rearward in the longitudinal direction A at a position away from the knife 2G in the radial direction R. Further, the sharp member 3G may be fixed not to move forward and rearward while protruding from the first opening 12a to the distal end side A1.

The main body portion 31G may be an elongated cylindrical member. Further, the “cylindrical shape” also includes a shape close to a cylindrical shape, in addition to a strict cylindrical shape. An outer diameter of the main body portion 31G may be greater than an inner diameter of the first region 121G and may be smaller than an inner diameter of the second region 122. In such an example, the main body portion 31G cannot be inserted through the first region 121G in the longitudinal direction A, and can move the second region 122 forward and rearward in the longitudinal direction A.

A fourth pipeline 36 may be formed in the main body portion 31G. The knife 2G may be inserted through the fourth pipeline 36. The knife 2G can move forward and rearward through the fourth pipeline 36 in the longitudinal direction A.

The flange 33G (e.g., a diameter expansion part) may be provided on the proximal end of the main body portion 31G. An outer diameter of at least a part of the flange 33G may be greater than an inner diameter of a proximal end opening 12b of the first through-hole 12G. In such an example, the flange 33G cannot be inserted through the first through-hole 12G. The distal end of the second operation wire 42 may be connected to the flange 33G of the sharp member 3G.

The distal end portion 34G can extend from the distal end of the main body portion 31G, and a cylindrical member may be divided in the longitudinal direction A to form a semi-cylindrical shape. That is, the distal end portion 34G may have a slit portion 34a formed in a semi-cylindrical shape. A part of the flange 21 can protrude outward from an outer circumferential surface and/or an inner circumferential surface of the main body portion 31G in the radial direction, and the flange 21 can move forward and rearward toward a distal side A1 other than a proximal end 34b of the slit portion 34a. In addition, the slit portion 34a does not need to have a semi-cylindrical shape, and for example, a groove having a width smaller than the inner diameter of the main body portion 31 may be formed in the longitudinal direction A.

The sharp portion 35G may be a member in which the distal end side A1 provided on the distal end of the distal end portion 34G is sharp. As shown in FIG. 30 and FIG. 31, the sharp portion 35G may be formed in a shape in which an edge 35a of the distal end side A1 of the semi-cylindrical shape is inclined with respect to the longitudinal direction A. A distal end 35b of the sharp portion 35G may be pointed toward the distal end side A1.

As shown in FIG. 35, the distal end portion 34G and the sharp portion 35G cannot inserted through the diameter reduction region 124 in the first region 121G, and can move forward and rearward in the diameter expansion region 125. In addition, a position of a maximum outer circumference of the proximal end surface 21b of the knife may be outside the inner circumferential surface of the diameter reduction region 124, and the flange 21 cannot be inserted through the diameter reduction region 124.

As shown in FIG. 34, when the sharp member 3G advances against the sheath 1G, a distal end 31a of the main body portion 31G of the sharp member 3G can come into contact with the step difference 12Gg of the distal end member 11G. As the distal end 31a of the main body portion 31G of the sharp member 3G comes into contact with the step difference 12Gg of the distal end member 11G, the distal end of the sharp member 3G may be positioned at the distal end position P4 that is a position closest to the distal end side A1. Further, as the distal end of the flange 33G of the sharp member 3G comes into contact with the proximal end opening 12b of the first through-hole 12G, the distal end of the sharp member 3G may be positioned at the distal end position P4.

The protrusion amount L3 of the sharp member 3G (a maximum protrusion amount of the sharp member 3G, see FIG. 34) protruding from the first opening 12Ga when the distal end of the sharp portion 35G is disposed at the distal end position P4 may be greater than the protrusion amount L2 of the knife 2G (a maximum protrusion amount of the knife 2G, see FIG. 33) protruding from the first opening 12Ga when the distal end of the knife 2E is disposed at the second position P2.

The liquid supply port 54 may be connected to a proximal end of the third pipeline 43 via the pipeline formed in the slider 52. The liquid supplied from the liquid supply port 54 can pass through the water supply flow path WR (the third pipeline 43, and the first pipeline 23) and can be discharged from the distal end opening 23a.

### [Variant (Treatment Tool 100H) Treatment Tool 100G]

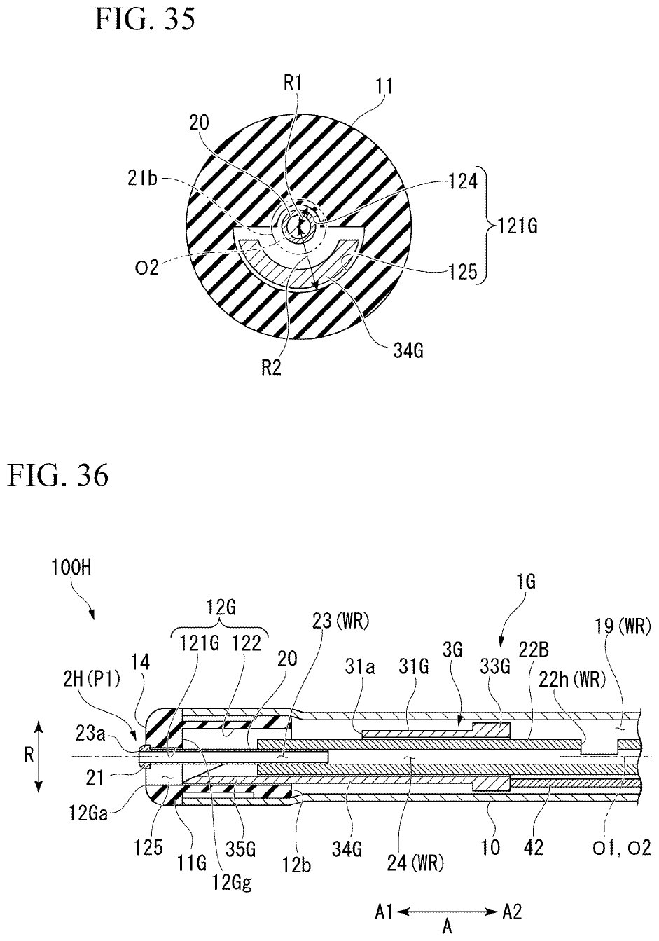

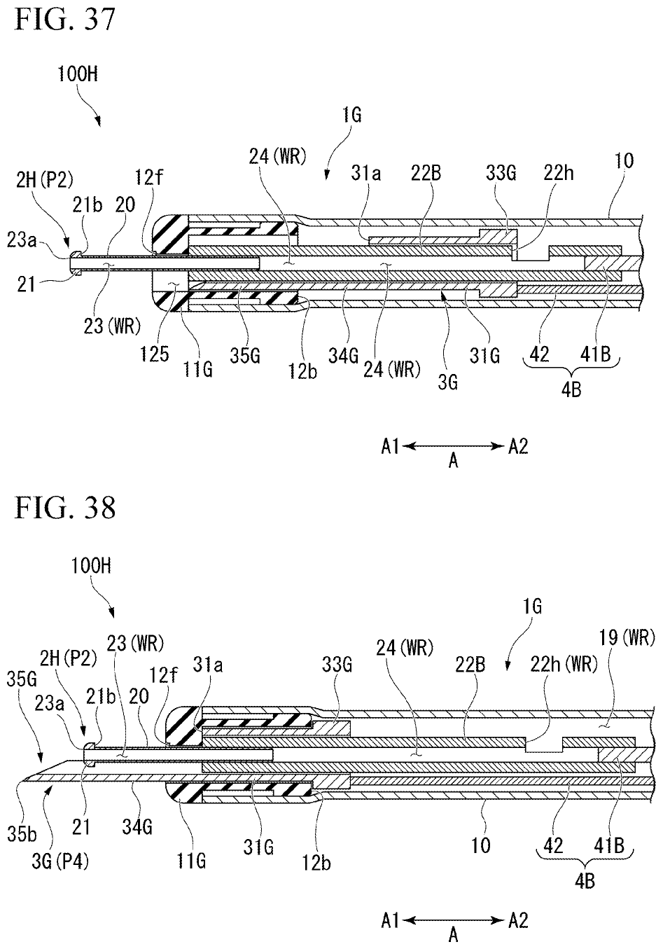

FIG. 36 to FIG. 38 are cross-sectional views showing a treatment tool 100H that is a variant of the treatment tool 100G. The treatment tool 100H is distinguished from the treatment tool 100G in the water supply flow path WR. The treatment tool 100H may include a sheath 1G, a knife 2H, a sharp member 3G, an operation wire 4B, and an operation unit 5. The knife 2H may include a knife main body 20, a flange 21, and a connecting member 22B.

The knife 2H may be inserted through the fourth pipeline 36. The knife 2H can move forward and rearward through the fourth pipeline 36 in the longitudinal direction A.

The liquid supply port 54 may be connected to a proximal end of the internal space 19 of the sheath 1G via the pipeline formed in the slider 52. The liquid supplied from the liquid supply port 54 can pass through the water supply flow path WR (the internal space 19 of the sheath 1G, the water supply port 22h, the second pipeline 24, and the first pipeline 23) and can be discharged from the distal end opening 23a.

According to the treatment tool 100G and the treatment tool 100H of the embodiment, incision or exfoliation treatment and local injection treatment can be appropriately performed, and the treatment tool 100G and 100H can be more easily handled by a user or operator.

Hereinafter, while the fourth embodiment has been described in detail with reference to the accompanying drawings, the specific configuration is not limited to the embodiment and may include design changes or the like without departing from scope of the present disclosure. In addition, the components shown in the above-mentioned embodiment and any variants can be configured by appropriately combining them.

### Fifth Embodiment

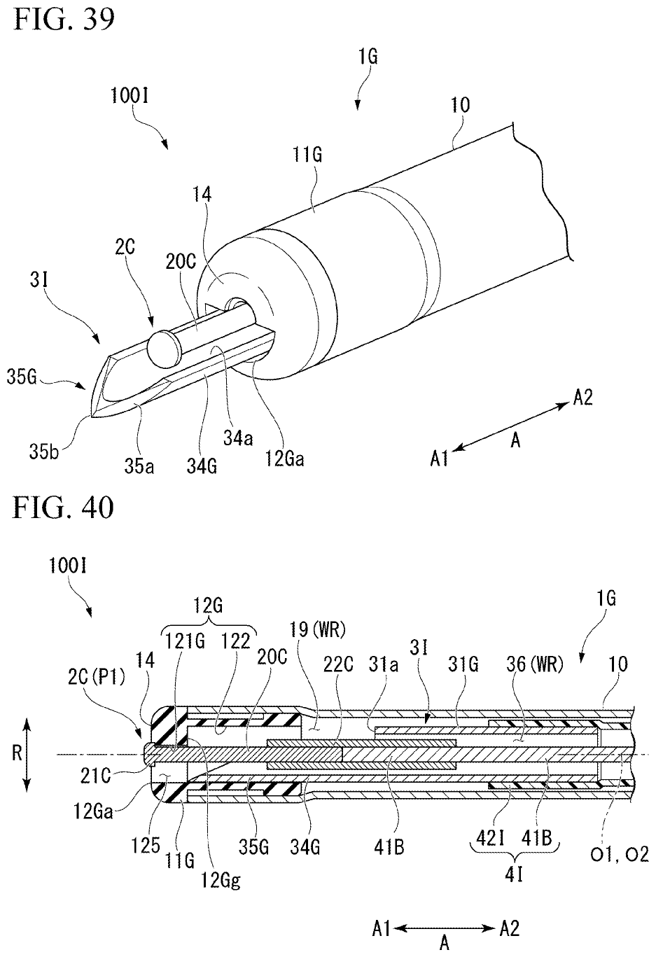

A treatment tool 100I according to a fifth embodiment will be described with reference to FIG. 39 to FIG. 42. In the following description, the common configurations described above are designated by the same reference signs and overlapping descriptions will be omitted.

FIG. 39 is a perspective view of a distal end portion of the treatment tool 100I.

The treatment tool (treatment tool for an endoscope) 100I may constitute an endoscope treatment system together with the endoscope 200, like the treatment tool 100 of the first embodiment. The treatment tool 100I may include a sheath 1G, a knife 2C, a sharp member (needle) 3I, an operation wire 41, and an operation unit 5.

FIG. 40 to FIG. 42 are cross-sectional views of a distal end portion of the treatment tool 100I.

As shown in FIG. 40, when the knife 2C is retracted against the sheath 1G, the proximal end surface 21b of the knife 2C may come into contact with a flange storage part 12f (e.g., a first surface) of the distal end member 11G. As the proximal end surface 21b of the knife 2C comes into contact with the flange storage part 12f of the distal end member 11G, the distal end of the knife 2C may be positioned at the first position P1 that is a position closest to the proximal end side A2. In a state in which the proximal end surface 21b of the knife 2C comes into contact with the flange storage part 12f of the distal end member 11G, the sharp member 3I can move forward and rearward with respect to the knife 2C in the longitudinal direction A. In such an example, the flange storage part 12f may be not formed in the first opening 12a. In this case, a distal end surface of the distal end member 11G is a first surface, and comes into contact with the proximal end surface 21b of the knife 2C.

The connecting member 22C may connect the knife main body 20C and the first operation wire 41B. An outer diameter of the connecting member 22C may be greater than an inner diameter of the first region 121G and may be smaller than an inner diameter of the second region 122. In such an example, the connecting member 22C cannot be inserted through the first region 121G in the longitudinal direction A, and can move forward and rearward through the second region 122 in the longitudinal direction A.

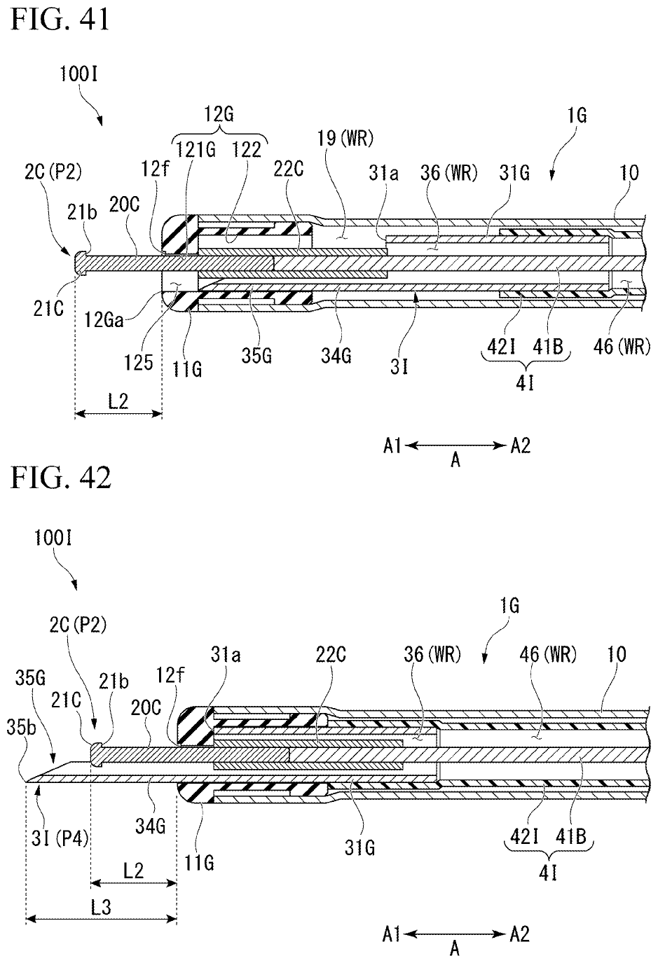

As shown in FIG. 41, when the knife 2C advances against the sheath 1G, the distal end of the connecting member 22C may come into contact with a step difference 12Gg (second surface) of the distal end member 11G. As the distal end of the connecting member 22C comes into contact with the step difference 12Gg of the distal end member 11G, the distal end of the knife 2C may be positioned at the second position P2 that is a position closest to the distal end side A1. In a state in which the connecting member 22C comes into contact with the step difference 12Gg of the distal end member 11G, the sharp member 3I can move forward and rearward with respect to the knife 2C in the longitudinal direction A.

The sharp member 3I may be formed of a resin material, a metal material, a ceramic material, or the like. The sharp member 3I may include a main body portion 31G, a distal end portion 34G, and a sharp portion 35G. In an example, the sharp member 3I does not have a flange 33G.

The knife 2C may be inserted through the fourth pipeline 36 of the sharp member 3I. The knife 2C can move forward and rearward through the fourth pipeline 36 in the longitudinal direction A.

As shown in FIG. 42, when the sharp member 3I advances against the sheath 1G, the distal end 31a of the main body portion 311 of the sharp member 3I may come into contact with the step difference 12Gg of the distal end member 11G. As the distal end 31a of the main body portion 31G of the sharp member 3I comes into contact with the step difference 12Gg of the distal end member 11G, the distal end of the sharp member 3I can be positioned at the distal end position P4 that is a position closest to the distal end side A1.

The operation wire 4I may be a wire inserted through an internal space (pipeline, lumen) 19 of the sheath 1G. The operation wire 4I may include a first operation wire 41B, and a second operation wire 42I.

The second operation wire 42I may be a wire configured to operate the sharp member 3I. The second operation wire 42I may be a hollow coil wire formed of a metal. The second operation wire 42I may be a tube. A distal end of the second operation wire 42I is fitted onto the main body portion 31G of the sharp member 3I. A proximal end of the second operation wire 42I may be connected to the lever 55 of the operation unit 5.

The protrusion amount L3 of the sharp member 3I (a maximum protrusion amount of the sharp member 3I, see FIG. 42) protruding from the first opening 12Ga when the distal end of the sharp portion 35G is disposed at the distal end position P4 may be greater than the protrusion amount L2 of the knife 2C (a maximum protrusion amount of the knife 2C, see FIG. 41) protruding from the first opening 12Ga when the distal end of the knife 2C is disposed at the second position P2.

The liquid supply port 54 may be connected to the proximal end of the second operation wire 42I via a pipeline formed in the slider 52. The liquid supplied from the liquid supply port 54 can pass through the water supply flow path WR (an internal space 46 of the second operation wire 42I, the fourth pipeline 36, the internal space 19 of the sheath 1G, and the first through-hole 12G) and can be discharged from the first opening 12Ga.

### [Variant (Treatment Tool 100J) of Treatment Tool 100I]

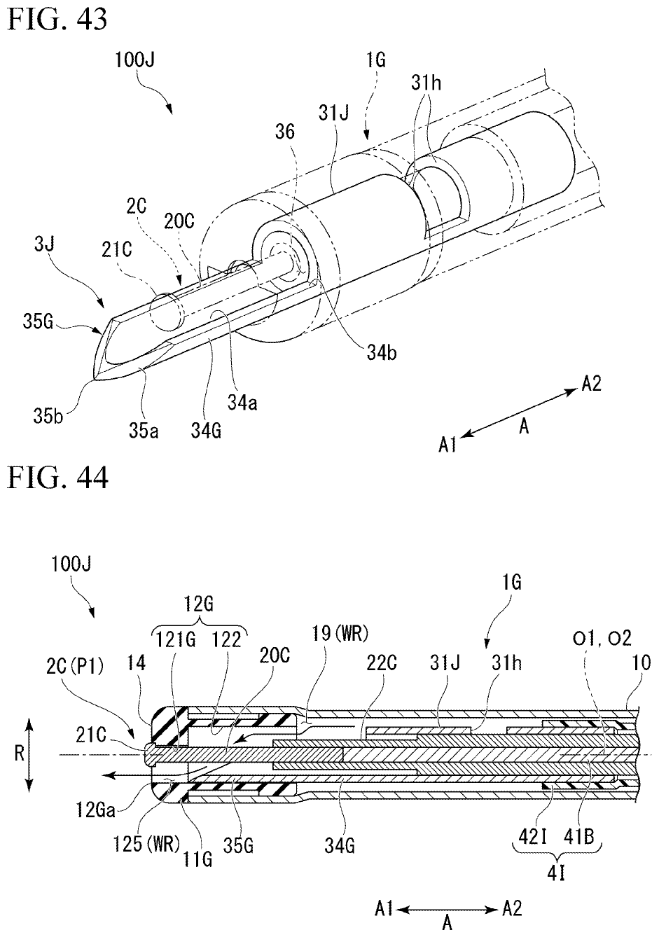

FIG. 43 is a perspective view showing a treatment tool 100J that is a variant of the treatment tool 100I. FIG. 44 to FIG. 46 are cross-sectional views of a distal end portion of the treatment tool 100J. The treatment tool 100J is distinguished from the treatment tool 100I in the water supply flow path WR. The treatment tool 100J may include a sheath 1G, a knife 2C, a sharp member 3J, an operation wire 4I, and an operation unit 5.

The sharp member 3J may be a member formed of a resin material, a metal material, a ceramic material, or the like. The sharp member 3J may include a main body portion 31J, a distal end portion 34G, and a sharp portion 35G.

The main body portion 31J may further include a water supply port 31h compared to the main body portion 31G. The water supply port 31h may be a hole formed from the fourth pipeline 36 in the radial direction R, and may open in an outer circumferential surface of the main body portion 31J.

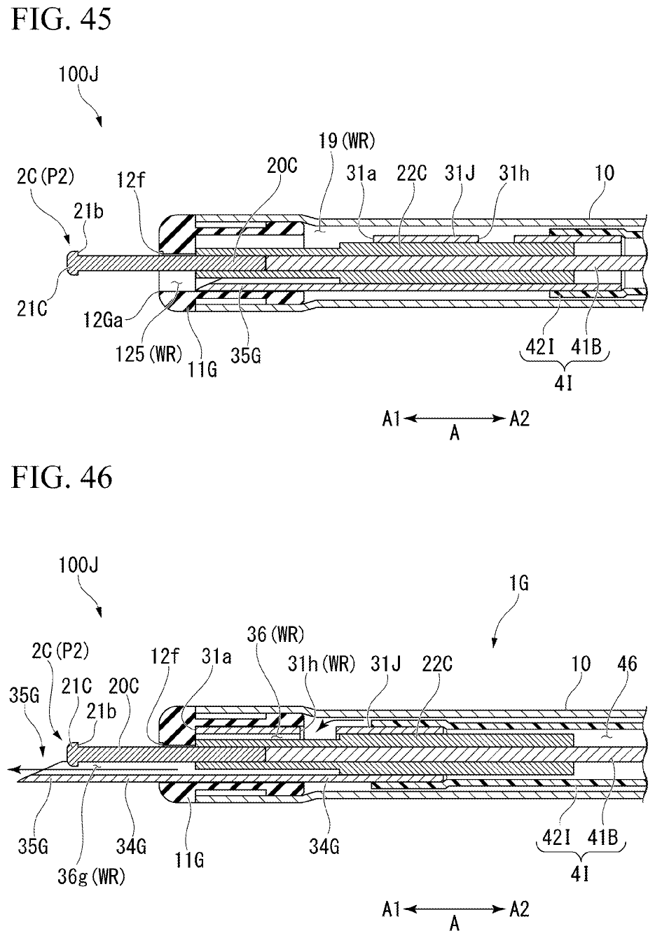

The liquid supply port 54 may be connected to a proximal end of the internal space 19 of the sheath 1G via a pipeline formed in the slider 52. As shown in FIG. 43 and FIG. 46, the liquid (fluid) supplied from the liquid supply port 54 can pass through the water supply flow path WR (the internal space 19 of the sheath 1G, the water supply port 31h, and the fourth pipeline 36) and can be discharged from the sharp portion 35G of the sharp member 3J. More specifically, as shown in FIG. 46, in a state in which the sharp portion 35G of the sharp member 3J and the knife 2C protrude from the first opening 12Ga (the sheath 1G), a gap 36g between the knife 2C and the sharp portion 35G of the sharp member 3J may form a part of the water supply flow path WR, and the liquid passing through the internal space 19 of the sheath 1G can pass through the gap 36g. Further, in the example shown in FIG. 44 and FIG. 45, in a state in which the sharp member 3J is accommodated in the sheath 1G, the liquid passing through the internal space 19 of the sheath 1G does not pass through the gap 36g and is discharged from the diameter expansion region 125.

According to the treatment tool 100I and the treatment tool 100J of the embodiment, incision exfoliation treatment and local injection treatment can be appropriately performed, the treatment tool 100I and 100J can be more easily handled by a user or operator.

Hereinabove, while the fifth embodiment has been described in detail with reference to the accompanying drawings, the specific configuration is not limited to the embodiment and may include design changes or the like without departing from the scope of the present disclosure. In addition, the components shown in the above-mentioned embodiment and variant can be configured by appropriately combining them.

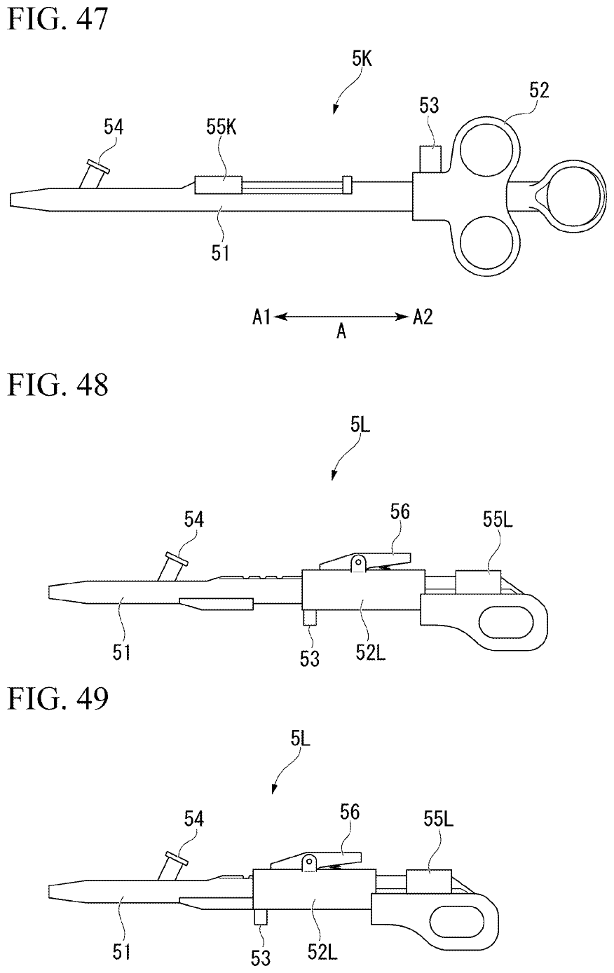

FIG. 47 is a view showing an operation unit 5K that is a variant of the operation unit 5.

The operation unit 5K may include an operation unit main body 51, a first slider 52, a feeding connector 53, a liquid supply port 54, and a second slider 55K.

The liquid supply port 54 may be provided on the operation unit main body 51. The liquid supply port 54 may be connected to a proximal end of the third pipeline 43 via the pipeline formed in the operation unit main body 51.

The second slider 55K may be movably attached to the operation unit main body 51 in the longitudinal direction A. The second slider 55K may be attached closer to the distal end side A1 than the first slider 52. A proximal end of the second operation wire 42 may be attached to the second slider 55K. As an operator moves the second slider 55K forward and rearward relative to the first slider 52, the second operation wire 42 and the sharp member 3 can move forward and rearward.

FIG. 48 and FIG. 49 are views showing an operation unit 5L that is a variant of the operation unit 5.

The operation unit 5L may include an operation unit main body 51, a first slider 52L, a feeding connector 53, a liquid supply port 54, a second slider 55L, and a switch 56.

The first slider 52L may be movably attached to the operation unit main body 51 in the longitudinal direction A. A proximal end of the first operation wire 41 may be attached to the first slider 52L. As an operator moves the first slider 52L forward and rearward relative to the operation unit main body 51, the first operation wire 41 and the knife 2 can move forward and rearward. When the operator releases his hand from the first slider 52L, the position of the first slider 52L with respect to the operation unit main body 51 may be fixed by a locking mechanism such as a ball click or the like.

The second slider 55L may be movably attached to the first slider 52L in the longitudinal direction A. The second slider 55L can move forward and rearward together with the first slider 52L, and further, can independently move forward and rearward with respect to the first slider 52L. A proximal end of the second operation wire 42 may be attached to the second slider 55L. As the operator moves the second slider 55L forward and rearward relative to the operation unit main body 51, the second operation wire 42 and the sharp member 3 can move forward and rearward.

The switch 56 may restrict forward and rearward movement of the first slider 52L and the second slider 55L in the longitudinal direction A. In such an example, when the switch 56 is pushed, forward and rearward movement of the first slider 52L and the second slider 55L is not restricted. When the switch 56 is not pushed, forward and rearward movement of the first slider 52L and the second slider 55L is restricted.

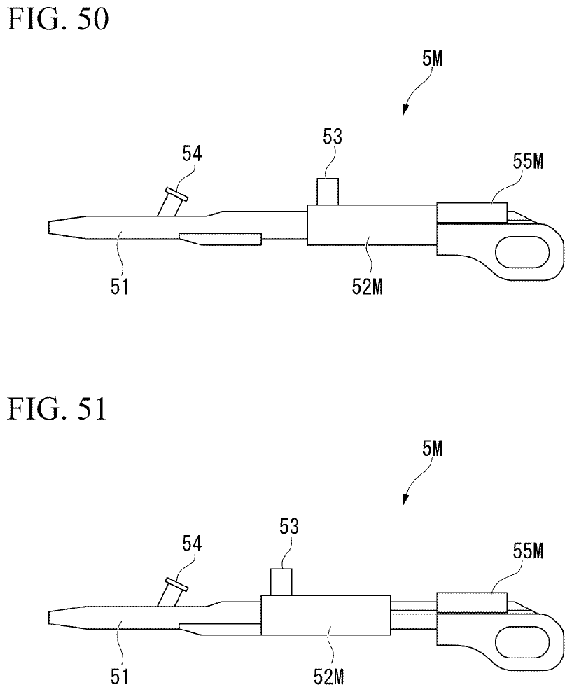

FIG. 50 and FIG. 51 are views showing an operation unit 5M that is a variant of the operation unit 5.

The operation unit 5M may include an operation unit main body 51, a first slider 52M, a liquid supply port 54, and a second slider 55M.

The first slider 52M may be movably attached to the operation unit main body 51 in the longitudinal direction A. A proximal end of the first operation wire 41 is attached to the first slider 52M. As the operator moves the first slider 52M forward and rearward relative to the operation unit main body 51, the first operation wire 41 and the knife 2 can move forward and rearward. When the operator releases his/her hand from the first slider 52M, the position of the first slider 52M with respect to the operation unit main body 51 can be fixed by a locking mechanism such as a ball click or the like.

The second slider 55M may be attached to the operation unit main body 51 to be movable in the longitudinal direction A independently from the first slider 52M. However, in a state in which the first slider 52M is retracted as shown in FIG. 50, the second slider 55M cannot move forward due to a contact with the first slider 52M. A proximal end of the second operation wire 42 may be attached to the second slider 55M. As the operator moves the second slider 55M forward and rearward relative to the operation unit main body 51, the second operation wire 42 and the sharp member 3 can move forward and rearward.

Further, the operation unit 5K, the operation unit 5L and the operation unit 5M, which are the variants of the operation unit 5, can be used in place of the operation unit 5 of the first to fifth embodiments.

The present disclosure can be applied to a treatment tool for an endoscope having a water supply function, or the like.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

What is claimed is:

1. A treatment tool for use with an endoscope, the treatment tool comprising:
a sheath having a distal end and a proximal end;
an electrode provided on a distal end portion of the sheath; and
a rod provided at a position away from the electrode in a radial direction of the electrode, the rod being advanceable and retractable with respect to the sheath, wherein, in a state in which at least the electrode and the rod protrude from the sheath, a part of a flow path through which a fluid passes is formed in at least a part of a space between the electrode and the rod.

2. The treatment tool according to claim 1, wherein the rod is configured to surround at least a part of the electrode in the radial direction of the electrode.

3. The treatment tool according to claim 1, wherein an outer diameter of the rod is smaller than a minimum outer diameter of a portion of the electrode exposed from a distal end of the sheath.

4. The treatment tool according to claim 1, wherein
the sheath includes a first opening formed in the distal end of the sheath and a second opening located away from the first opening in the radial direction, the electrode is configured to pass through the first opening, and the rod is configured to be advanceable and retractable with respect to the sheath through the second opening.

5. The treatment tool according to claim 1,
wherein the sheath includes a first opening formed in the distal end of the sheath,
wherein the electrode and the rod are configured to pass through the first opening of the distal end of the sheath,
wherein a the electrode and the rod are configured to protrude from the sheath and accommodate in the sheath,
wherein the rod includes a needle, and
wherein at least a part of the flow path is formed between the electrode and a distal end of the needle.

6. The treatment tool according to claim 4, wherein a distal end of the electrode is configured to pass through the first opening of the distal end of the sheath, the distal end of the electrode being configured to protrude from the sheath and accommodate in the sheath.

7. The treatment tool according to claim 4, wherein a distal end of the rod is configured to pass through the second opening of the distal end of the sheath, the distal end of the rod being configured to protrude from the sheath and accommodate in the sheath.

8. The treatment tool according to claim 7, wherein an outer diameter of the rod is smaller than a minimum outer diameter of a portion of the electrode exposed from the distal end of the sheath.

9. A treatment tool for use with an endoscope comprising:
a sheath having a distal end and a proximal end;
an electrode provided on a distal end portion of the sheath; and
a rod provided at a position away from the electrode in a radial direction of the electrode, the rod being advanceable and retractable with respect to the sheath, wherein an outer diameter of the rod is smaller than a minimum outer diameter of a portion of the electrode exposed from the distal end of the sheath.

10. The treatment tool according to claim 9, wherein:
the sheath includes a first opening formed in the distal end of the sheath and a second opening located away from the first opening in the radial direction, the electrode is configured to pass through the first opening, and wherein the rod is configured to be advanceable and retractable with respect to the sheath through the second opening.

11. The treatment tool according to claim 10, wherein a distal end of the electrode is configured to pass through the first opening of the distal end of the sheath, the distal end of the electrode being configured to protrude from the sheath and accommodate in the sheath.

12. The treatment tool according to claim 10, wherein a distal end of the rod is configured to pass through the second opening of the distal end of the sheath, the distal end of the rod being configured to protrude from the sheath and accommodate in the sheath.

13. The treatment tool according to claim 10, wherein at least a part of a flow path through which a fluid passes is formed in at least a part of a space between the electrode and the rod.

14. The treatment tool according to claim 13, wherein a gap is provided between the rod and an inner circumferential surface of the second opening, and wherein at least a part of the flow path includes at least a part of the gap.

15. The treatment tool according to claim 10, wherein the distal end portion of the sheath has a distal end member formed of an insulating material, wherein the first opening and the second opening are formed in the distal end member, and wherein
the rod includes a flange that abuts the distal end member in a state in which a distal end of the rod protrudes from the second opening.

16. The treatment tool according to claim 15, wherein a protrusion amount where the rod protrudes from the second opening when the flange abuts the distal end member is greater than a protrusion amount where the electrode maximally protrudes from the first opening.

17. The treatment tool according to claim 15, wherein a protrusion amount where the rod protrudes from the second opening when the flange abuts the distal end portion is smaller than a protrusion amount where the electrode maximally protrudes from the first opening.

18. A treatment tool for use with an endoscope comprising:
a sheath having a distal end and a proximal end;
an electrode provided on a distal end portion of the sheath; and
a rod configured to be advanceable and retractable,
wherein the distal end portion of the sheath includes a groove that is a flow path, and
wherein the electrode is disposed between the groove and the rod.

19. The treatment tool according to claim 18, wherein a distal end of the rod is configured to protrude from the sheath and accommodate in the sheath.

20. The treatment tool according to claim 18,
wherein the electrode includes a flange, and
wherein the groove does not overlap the flange.
